(19) United States
(12) Patent Application Publication
(10) Pub. No.: US 2025/0201343 A1
(43) Pub. Date: Jun. 19, 2025

(54) PREDICTING PATIENT RESPONSE TO CANCER THERAPY VIA HISTOPATHOLOGY IMAGES

(71) Applicants: PANGEA BIOMED LTD, Tel Aviv (IL); AUSTRALIAN NATIONAL UNIVERSITY, ACT, Australian Capital Territory (AU); The United States of America, as represented by the Secretary, Department of Health and Human Servic, Bethesda, MD (US)

(72) Inventors: Danh-Tai HOANG, Holt, ACT (AU); Eric STONE, Griffith, ACT (AU); Eytan RUPPIN, Rockville, MD (US); Gal DINSTAG; Ranit AHARONOV, Ramat Hasharon (IL); Tuvik BEKER, Tel Aviv (IL)

(73) Assignees: PANGEA BIOMED LTD, Tel Aviv (IL); AUSTRALIAN NATIONAL UNIVERSITY, Canberra, ACT (AU); The United States of America, as represented by the Secretary, Department of Health and Human Servic, Bethesda, MD (US)

(21) Appl. No.: 18/868,028

(22) PCT Filed: Jun. 6, 2023
(86) PCT No.: PCT/US2023/024571

§ 371 Date: Nov. 21, 2024

Related U.S. Application Data
(60) Provisional application No. 63/349,829, filed on Jun. 7, 2022.

(51) Int. Cl. G16B 25/10; G06N 3/0455; G06N 3/0464; G06N 20/00; G16B 40/20; G16H 20/10; G16H 30/40; G16H 50/70

(52) U.S. Cl. CPC G16B 25/10; G06N 3/0455; G06N 3/0464; G06N 20/00; G16B 40/20; G16H 20/10; G16H 30/40; G16H 50/70

(57) ABSTRACT

A method of training an artificial neural network to predict gene expression for a patient having a pathological condition is provided. The method comprises obtaining a set of histopathological images from a database of patients having the pathological condition, collecting from the database a set of gene expression profiles corresponding to the histopathological images, identifying in the set of gene expression profiles a set of considered genes, selecting a training subset of the considered genes comprising genes characterized by similar median gene expression values, and training the neural network to predict a gene expression value of an input histopathological image, using gene expression profiles of the genes in the training subset simultaneously.

### GOVERNMENT INTEREST STATEMENT

This invention was made in part with Government support under project number ZIA BC 011802 by the National Institutes of Health, National Cancer Institute. The Government has certain rights in this invention.

### TECHNOLOGICAL FIELD

The presently disclosed subject matter relates to systems and methods for predicting a gene expression based on a digital histopathological image, in particular to systems and methods in which a trained artificial neural network is used for the prediction.

### BACKGROUND

Histopathology has long been considered the gold standard of clinical diagnosis and prognosis in cancer. Recently, molecular markers such as tumor gene expression have proven increasingly valuable for enhancing diagnosis and precision oncology. Digital histopathology has been explored to combine these complementary sources of information using machine learning, artificial intelligence, and big data.

Whole slide images (WSI) of tissue stained with haematoxylin and eosin have been used to computationally diagnose tumors, classify cancer types, distinguish tumors with low or high mutation burden, identify genetic mutations, predict patient survival, detect DNA methylation patterns and mitoses, and quantify tumor immune infiltration.

### SUMMARY

According to an aspect of the presently disclosed subject matter, provided herein is a method of predicting a gene expression profile in a subject based on a histopathological image using an artificial neural network (ANN), the method comprising:
(a) providing an artificial neural network (ANN) comprising a feature extraction module, an auto-encoder, and a multi-layer perceptron (MLP),
   wherein said extraction module is configured to receive an input signal representing a histopathological image, to extract a predetermined number of features from said input signal, and to transmit said extracted features to said autoencoder;
   wherein said autoencoder is configured to receive the features extracted from the extraction module, to compress the number of said extracted features, and to present said compressed features to said MLP;
   wherein said MLP is configured to receive the compressed features from the autoencoder in an input layer of neurons and to provide an output in an output layer of neurons, said output representing a gene expression profile;
   wherein said ANN is trained to predict a gene expression profile from a histopathological image;
(b) providing said histopathological image to said ANN;
(c) obtaining a predicted gene expression profile of the subject with said ANN.

According to an aspect of the presently disclosed subject matter, provided herein is a method of predicting the response to a therapeutic intervention in a subject having a pathological condition, the method comprising the steps of:
(a) providing one or more histopathological images from said subject;
(b) obtaining a predicted gene expression profile of said subject according to the method described above;
(c) determining a treatment score from the predicted gene expression profile for the subject with a SELECT or an ENLIGHT algorithm, wherein the treatment score is indicative of a likelihood of successfully treating said subject with said therapeutic intervention; and
(d) predicting the response to said therapeutic intervention in said subject based on said treatment score.

According to an aspect of the presently disclosed subject matter, provided herein is a method of selecting a therapeutic intervention for a subject in need thereof, the method comprising the steps of:
(a) providing a plurality of therapeutic interventions;
(b) predicting the response to each of said therapeutic interventions in said subject according to method provided above; and
(c) selecting the therapeutic intervention with the treatment score that is indicative of the highest likelihood of successfully treating the subject.
(d) According to an aspect of the presently disclosed subject matter, provided herein is a method of treating a subject in need thereof, the method comprising:
(e) selecting a therapeutic intervention for said subject according to the method provided above; and
(f) treating the subject with the therapeutic intervention selected in step (a).

According to an aspect of the presently disclosed subject matter, provided herein is a method of ranking a set of subjects having a pathological condition based on the likelihood of successfully treating each subject with a therapeutic intervention, the method comprising the steps of:
(a) providing a set of subjects having a pathological condition;
(b) providing one or more histopathological images of each of said subjects;
(c) predicting the response to said therapeutic intervention in each subject according to the method provided above; and
(d) ranking each subject in the set of subjects based on the probability of successfully treating that subject with said therapeutic intervention.

In some related aspect, the set of subjects are prospective participants in a clinical trial.

According to an aspect of the presently disclosed subject matter, provided herein is a method for designing a clinical trial for a therapeutic intervention, the method comprising the steps of:
(a) providing a set of subjects having a pathological condition;
(b) predicting the response of each of said subjects to said therapeutic intervention according to the methods provided above;
(c) selecting for said clinical trials the subjects having a treatment score higher than a predetermined value.

According to an aspect of the presently disclosed subject matter, provided herein is a method of monitoring a subject having a pathological condition who is or will be undergoing a therapeutic intervention, the method comprising the steps of:
(a) predicting the response to said therapeutic intervention in said subject according to the method described above;
(b) repeating steps (a) a plurality of times to monitor the course of treatment.

In some related aspect, the ANN is trained to predict a gene expression profile from a histopathological image by a training method comprising:
(a) providing a training set of histopathological images taken from patients having a pathological condition;
(b) providing a training set of gene expression profiles, wherein each gene expression profile from said training set of gene expression profiles corresponds to one of the histopathological images from said training set of histopathological images;
(c) presenting to said feature extraction module a first input signal representing a histopathological image from the training set of histopathological images and presenting to said output layer of said MLP a second input signal representing the corresponding gene expression profile;
(d) adjusting the weights of said ANN; and
(e) repeating steps (c) and (d) for all histopathological images and their corresponding gene expression profile of said training sets;
(f) repeating steps (c)-(e) a number of epochs.

In some related aspect, each gene expression profile from said training set is divided into subsets of genes characterized by similar median expression, and wherein said ANN is trained with each subset separately. In some related aspect, the training comprises a hold-out method. In some related aspect, the training comprises k-fold cross-validation.

In some related aspect, the histopathological image is divided into sub-images, and wherein each sub-image is presented separately to the input layer of said feature extraction module. In some related aspect, the predicted gene expression of a histopathological image is calculated by pooling the gene expression profile predicted for each of said sub-images. In some related aspect, the pooling comprises the average or the median of said output produced by each of said sub-images.

In some related aspect, the ANN comprises a convolutional neural network (CNN). In some related aspect, the CNN comprises a ResNet50 CNN. In some related aspect, the number of neurons in the input layer of the MLP is equal to the number of features compressed by the autoencoder. In some related aspect, the MLP comprises a hidden layer, and the number of neurons in said hidden layer is less than or equal to the number of features compressed by the autoencoder.

In some related aspect, the number of neurons in the output layer of the MLP is equal to the number of genes of said gene expression profile. In some related aspect, the number of features compressed by the autoencoder is no greater than about half, or no greater than about one quarter of the number of features extracted by the ANN.

In some related aspect, the ANN is configured to extract 2,048 features, and the autoencoder is configured to extract 512 features. In some related aspect, said histopathological images are whole slide images of stained tissue. In some related aspect, said tissue is stained with haematoxylin and eosin.

In some related aspect, said gene expression profile comprises genes expressed above a predetermined threshold. In some related aspect, said genes above a predetermined threshold are identified using edgeR. In some related aspect, the pathological condition is a cancer. In some related aspect, the cancer is selected from a group including breast, lung, brain, kidney, colorectal, prostate, gastric, head and neck, cervical, and pancreas cancer.

In some aspects, disclosed herein is a method for identifying a pair of genes comprising a synthetic lethality (SL), synthetic rescue (SR), or synthetic dosage lethality (SDL) interaction, using a depletion model, said method implemented by a computer processor executing program instructions comprising:
(a) obtaining gene expression profiles from a set of subjects according to the methods described above;
(b) transforming expression data relating to each of two genes from said gene expression profiles, through ranking, thereby producing two uniform transformed distributions in the range [0, 1];
(c) calculating a resulting joint expression distribution for the gene pair, having uniform marginal distributions;
(d) identifying a parametric family of distributions comprising a shape parameter wherein said shape parameter determines the degree of corner depletion or enrichment for one or more of the corners in the joint distribution, and fitting said shape parameter to said joint expression data; and
(e) calculating a value of a best-fitting shape parameter as an indication of the genetic interaction between said two genes.

In some aspects, disclosed herein is a method for identifying a pair of genes comprising a synthetic lethality (SL), synthetic rescue (SR), or synthetic dosage lethality (SDL) interaction, using a parametric survival model, said method implemented by a computer processor executing program instructions comprising:
(a) obtaining gene expression profiles from a set of subjects according to the method of claim 1;
(b) Transforming expression data relating to each of two genes from a population, thereby producing two uniform transformed distributions in the range [0, 1];
(c) Calculating a resulting joint expression distribution for the gene pair, having uniform marginal distributions;
(d) Identifying a theoretical distribution function D that approximates the joint distribution of the transformed expression levels of said pair of genes;
(e) Calculating a covariate value (c(p)) for a given patient in a population of patients (cohort P), by calculating a ratio of the density of said theoretical distribution function D at joint expression values (x,y) of said gene pair, to a maximal D density value or a minimal D density value, across a full joint distribution space; and
(f) Assessing a correlation between (i) a set of covariates C:={c{p)|p in P} obtained in “c” for said patients of cohort P; and (ii) survival of the patients in said cohort, as an assessment of the strength of the corresponding genetic interaction between said gene pair.

In some related aspect, the synthetic rescue (SR) comprises synthetic rescue DD (SR-DD) or synthetic rescue DU (SR-DU).

In some aspects, disclosed herein is a method for identifying a drug target candidate, the method comprising identifying genes comprising SL, SR, or SDL interactions according to the methods disclosed above; wherein a gene, or a protein encoded thereof, comprising a number of SL, SR, or SDL interactions above a predetermined threshold, is as a drug target candidate.

In some aspects, disclosed herein is a method of predicting the efficiency of novel therapeutic combinations, the method comprising identifying pair of genes comprising SL, SR, or SDL interactions according to the methods disclosed above; wherein a therapeutic combination targeting a pair of genes comprising SL, SR, or SDL interactions is predicted to be efficient.

In some aspects, disclosed herein is a method of training an artificial neural network (ANN) to predict a gene expression profile of a patient having a pathological condition, the method comprising:
(a) providing an artificial neural network (ANN) comprising a feature extraction module, an auto-encoder, and a multi-layer perceptron (MLP),
   wherein said extraction module is configured to receive an input signal representing a histopathological image, to extract a predetermined number of features from said input signal, and to transmit said extracted features to said autoencoder;
   wherein said autoencoder is configured to receive the features extracted from the extraction module, to compress the number of said extracted features, and to present said compressed features to said MLP;
   wherein said MLP is configured to receive the compressed features from the autoencoder in an input layer of neurons and to provide an output in an output layer of neurons, said output representing a gene expression profile;
(b) providing a training set of histopathological images taken from patients having said pathological condition;
(c) providing a training set of gene expression profiles, wherein each gene expression profile from said training set of gene expression profiles corresponds to one of the histopathological images from said training set of histopathological images;
(d) presenting to said feature extraction module a first input signal representing a histopathological image from the training set of histopathological images and presenting to said output layer of said MLP a second input signal representing the corresponding gene expression profile;
(e) adjusting the weights of said ANN; and
(f) repeating steps (d) and (e) for all histopathological images and their corresponding gene expression profile of said training sets;
(g) repeating steps (d)-(f) a number of epochs.

In some related aspect, each of said histopathological images is divided into sub-images, and each sub-image is used as the input to the feature extraction module to train the ANN. In some related aspect, wherein said sub-images are non-overlapping. In some related aspect, sub-images in which at least about 50% of the pixels do not represent an image of tissue are excluded.

In some aspects, disclosed herein is a system for predicting a gene expression profile based on a histopathological image, the system comprising:
(a) an artificial neural network (ANN), comprising a feature extraction module, an auto-encoder, and a multi-layer perceptron (MLP),
(b) wherein said extraction module is configured to receive an input signal representing a histopathological image, to extract a predetermined number of features from said input signal, and to transmit said extracted features to said autoencoder;
(c) wherein said autoencoder is configured to receive the features extracted from the extraction module, to compress the number of said extracted features, and to present said compressed features to said MLP;
(d) wherein said MLP is configured to receive the compressed features from the autoencoder in an input layer of neurons and to provide an output in an output layer of neurons, said output representing a gene expression profile;
(e) wherein said ANN is trained to predict a gene expression profile from a histopathological image.

In some aspects, disclosed herein is a system for predicting the response to a therapeutic intervention in a subject having a pathological condition, the system comprising the system for predicting a gene expression profile described above;
(a) wherein the system for predicting the response to a therapeutic intervention is configured to determine a treatment score from the predicted gene expression profile for the subject with a SELECT or an ENLIGHT algorithm, wherein the treatment score is indicative of a likelihood of successfully treating the subject with the therapeutic intervention; and
(b) wherein the system for predicting the response to a therapeutic intervention is configured to predict the response to said therapeutic intervention in said subject based on said treatment score.

In some aspects, provided herein is a system for selecting a therapeutic intervention for a subject in need thereof, the system comprising the system for predicting the response to a therapeutic intervention described above;
(a) wherein the system for selecting a therapeutic intervention is configured to provide a prognosis score for a plurality of therapeutic interventions; and
(b) wherein the system for selecting a therapeutic intervention is configured to select the therapeutic intervention with the treatment score that is indicative of the highest likelihood of successfully treating the subject.

In some aspects, disclosed herein is a system for ranking a set of subjects having a pathological condition based on the likelihood successfully treating each subject with a therapeutic intervention, the system comprising the system for predicting the response to a therapeutic intervention described above;
(a) wherein the system for ranking a set of subjects is configured to predict the response to a therapeutic intervention for each subject treated with said therapeutic intervention;
(b) wherein the system for ranking a set of subjects is configured to rank each subject from the set of subjects based on the probability of successfully treating that subject with said therapeutic intervention.

In some aspects, disclosed herein is a system for designing a clinical trial for a therapeutic intervention, the system comprising the system for predicting the response to a therapeutic intervention described above;
(a) wherein the system for designing a clinical trial is configured to receive information regarding a set of subjects having a pathological condition;
(b) wherein the system for designing a clinical trial is configured to select for said clinical trial subjects having a treatment score higher than a predetermined value.

In some aspects, disclosed herein is a system for monitoring a subject having a pathological condition who is or will be undergoing a therapeutic intervention, the system comprising the system for predicting the response to a therapeutic intervention described above; wherein the system for monitoring a subject is configured to predict the response to said therapeutic intervention in said subject a plurality of times.

In some aspects, disclosed herein is a system for training an artificial neural network (ANN) to predict gene expression profile of a patient having a pathological condition, the system comprising:
an artificial neural network (ANN), comprising a feature extraction module, an auto-encoder, and a multi-layer perceptron (MLP),
   wherein said extraction module is configured to receive an input signal representing a histopathological image, to extract a predetermined number of features from said input signal, and to transmit said extracted features to said autoencoder;
   wherein said autoencoder is configured to receive the features extracted from the extraction module, to compress the number of said extracted features, and to present said compressed features to said MLP;
   wherein said MLP is configured to receive the compressed features from the autoencoder in an input layer of neurons and to provide an output in an output layer of neurons, said output representing a gene expression profile;
   wherein said system is configured to
      (a) receive a training set of histopathological images taken from patients having said pathological condition;
      (b) receive a training set of gene expression profiles, wherein each gene expression profile from said training set of gene expression profiles corresponds to one of the histopathological images from said training set of histopathological images;
      (c) present to said feature extraction module a first input signal representing a histopathological image from the training set of histopathological images and presenting to said output layer of said MLP a second input signal representing the corresponding gene expression profile;
      (d) adjust the weights of said ANN; and
      (e) repeat steps (c) and (d) for all histopathological images and their corresponding gene expression profile of said training sets;
      (f) repeat steps (c)-(e) a number of epochs.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 schematically illustrates a system according to the presently disclosed subject matter for predicting the expression of each of a plurality of genes, based on a histopathological image input thereto.

FIG. 2 schematically illustrates an autoencoder of the system illustrated in FIG. 1.

FIG. 3 schematically illustrates a multilayer perceptron of the system illustrated in FIG. 1.

FIG. 4: Study overview. FIG. 4A shows the three main components of DeepPT's architecture: The pre-trained ResNet50 CNN unit extracts histopathology features from tile images. The autoencoder compresses the 2,048 features to a lower dimension of 512 features. The multi-layer perceptron integrates these histopathology features to predict the sample's gene expression. FIG. 4B shows an overview of the ENLIGHT pipeline: ENLIGHT starts by inferring the genetic interaction partners of a given drug from various cancer in-vitro and clinical data sources. Given the SL and SR partners and the transcriptomics for a given patient sample, ENLIGHT computes a drug matching score that is used to predict the patient response. Here, ENLIGHT uses DeepPT predicted expression to produce drug matching scores for each patient studied. FIG. 4C shows that DeepPT was trained with formalin-fixed paraffin-embedded (FFPE) slide images and their matched transcriptomics of TCGA patients from four cancer types, including breast, kidney, lung, and brain. After the training phase, the models were then applied to predict gene expression on the four internal (held-out) TCGA datasets and on two independent datasets on which it was never trained on. The predicted tumour transcriptomics served as input to ENLIGHT for predicting patient's response to treatment.

FIG. 5: Model architecture in detail. (a) The feature compression subnetwork consists of an input layer of 2,048 neurons, a bottleneck of 512 neurons, and an output layer of 2,048 neurons. (b) The MLP regression subnetwork consists of an input layer of 512 neurons, a hidden layer of 512 neurons, and an output layer with the number of neurons reflecting the number of genes in each group.

FIG. 6: The number of significantly predicted genes for each TCGA cohort, in comparison with the current state-of-the-art method, HE2RNA. For apples-to-apples comparison against HE2RNA, the performance of each cancer subtypes in Kidney (KIRC, KIRP, KICH) and Lung (LUSC, LUAD) are shown together.

FIG. 7: Histograms of the Pearson correlation coefficients between predicted and actual expression for each gene across test sets, for 16 TCGA cohorts (light blue) and 2 external cohorts (gray). Red dashed lines represent the correlation coefficient level beyond which the results are significant (p-value<0.05 after correction for multiple hypotheses testing).

FIG. 8: The number of significantly predicted genes, averaging over 30 randomly selected subsets. Each subset comprises 200 samples that were randomly selected from the cohort. Only cohorts with at least 200 samples were analyzed. Error bars represent standard error of the mean.

FIG. 9: Difference between histopathological features extracted from TCGA-Breast tiles and TransNEO-Breast tiles. UMAP visualization of 2,048 histopathological features that were extracted by using pre-trained ResNet50 CNN. 4,000 image tiles from each dataset were selected randomly to illustrate. Each point represents each feature vector of one image tile.

FIG. 10: The number of significantly predicted genes in two independent test cohorts, obtained by using pre-trained models on the corresponding TCGA cohorts.

FIG. 11: Pathway enrichment analysis on the significantly predicted genes. Each row represents a different cancer hallmark and each column a different cohort (the two right columns correspond to the two external cohorts). Values denote the multiple hypothesis corrected p-value for pathway enrichment among the genes significantly predicted by DeepPT.

FIG. 12: Comparison of the correlation of survival association in terms of log(HR) for three proliferation signatures (left: MK67; middle: Proliferation index; right: EMT pathway) based on actual (X axis) and predicted expressions (Y axis). Each point represents a different TCGA cohort, and points are color-coded according to the significance of survival association using a corrected p<0.05 cutoff: green denotes that the survival association was significant by both the actual and predicted signatures, red/black only by the actual/predicted signatures, respectively. Pearson R and corresponding p-values are denoted in each panel.

FIG. 13: A comparison between the performance of ENLIGHT-DeepPT when using the same methodology described in Dinstag et al. Med. 4(1):15-30.e8 to generate genetic interaction networks that constitutes ENLIGHT's predictive biomarkers (orange bars) and a revised methodology (blue bars) where we restricted ENLIGHT's biomarker to only include genes that showed significant positive correlation (corrected p<0.05) between actual and DeepPT-predicted values among the respective TCGA cohort (that is, according to the cancer type of each of the five drug response datasets). Left panel: Odds Ratio (OR) for each dataset, using the same clinical decision threshold that has been previously established in Dinstag et al. Med. 4(1):15-30.e8. Right panel: Average Precision (AP) for each dataset.

FIG. 14: Predicting treatment response from H&E slides. (a) Odds Ratio (OR, Y axis) for the five datasets tested and the aggregate cohort of all patients together (X axis). Drug and sample sizes are denoted in the X axis labels. Orange horizontal dashed line denotes an OR of 1 which is expected by chance. Bars are color coded according to the indication(s) of the respective cohort. Asterisks denote significance of OR being larger than 1 according to Fisher's exact test (b) Average Precision (AP, Y axis) for the five datasets and the aggregate cohort, as in a. Black horizontal dashed lines denote the ORR for each dataset. An AP higher than the ORR demonstrates better accuracy than expected by chance. Asterisks denote significance of AP being higher than response rate using one-sided proportion test. (c) OR of the Direct Supervised method (Y axis) for all 234 patients as a function of the fraction of patients above a given threshold (coverage, X axis). We present only coverage between 10-90% to avoid the measurement noise of extreme coverage values, where data is too small. Orange dashed line denotes the OR of ENLIGHT-DeepPT for all 234 patients at its original clinical decision threshold. The square denotes the threshold on the Direct Supervised that yields the same coverage as ENLIGHT-DeepPT at its original, fixed threshold. (d) Comparison of the OR of ENLIGHT-DeepPT and the Direct Supervised methods (Y axis) at thresholds that yield the same coverage (X axis). (e) Average Precision of ENLIGHT-DeepPT (cyan) and Direct Supervised (purple) for each dataset and on aggregate as in b. Dashed lines denote the ORR for each case as in b (f) OR for ENLIGHT-actual and ENLIGHT-DeepPT when predicting response to Trastuzumab (for the Trastuzumab1 cohort). (g) Comparison of AP (Y axis) for both ENLIGHT based models and the Sammut-ML predictor of Sammut et al. All methods were applied to the same patient group. Black horizontal dashed line denotes the ORR. All p-values were FDR corrected. *=p<0.1, **=p<0.05.

FIG. 15: Comparison of the predictive performance of ENLIGHT-DeepPT (orange bars) and the respective drug target(s) expression models (blue bars) for each patient cohort and on the aggregation of all patients. Left panel: Odd ratio (OR) for each cohort for the same thresholds established in [1]: Average Precision (AP) for each cohort.

FIG. 16: Improving patient stratification and clinical trial design for Bintrafusp Alfa. FIG. 18A shows the ENLIGHT matching score (EMS) of Bintrafusp Alfa calculated using ENLIGHT-DeepPT, depicted separately for responders and non-responders. FIG. 18B shows the sensitivity and PPV of cases that were matched to Bintrafusp Alfa by ENLIGHT-DeepPT versus overall response rate observed in the dataset. FIG. 18C shows the NPV (percentage of true non-responders out of those predicted as non-responders) as a function of the percentage of patients excluded. The horizontal line denotes the actual percentage of non-responders in the Bintrafusp Alfa cohort (i.e., the NPV expected by chance). FIG. 18D shows the response rate among the remaining patients (y axis) after excluding a certain percentage of the patients (x axis). The horizontal line denotes the overall response rate in the Bintrafusp Alfa cohort. The dotted-dashed line represents the upper bound on the response rate, achieved by excluding only true non-responders.

FIG. 17: Training strategies and their performance. (a) In the ensemble learning strategy (bagging), five models were trained independently with five internal training-validation splits; these five model predictions were averaged to make the final prediction. (b) In the model selection strategy, the “best” model with the highest performance on the validation set was chosen to make prediction on the test set. (c) Number of genes with mean correlation over 5 folds greater than a certain threshold, obtained from these strategies. The TCGA-Breast cohort was selected as an example. Note that with either strategy, DeepPT outperforms the current state-of-the-art approach, HE2RNA.

FIG. 18: Histograms of the number of tiles per slide by cohort. The number of tiles in each slide image from TCGA and NCI-Brain datasets ranges from 100 to 8,000 (a, b, c, d), while the number of tiles in each TransNEO-Breast slide image is much smaller, ranging from 100 to 1,000 (e).

FIG. 19: Histogram of median expression over slides. The median expression over samples of each gene commonly varies from 10 to 100,000 for every dataset considered in this study.

FIG. 20: Histograms of median and standard deviation of ResNet features (upper) and AutoEncorder features (lower); Median (left panels) and standard deviation values (right panels) are shown. The TCGA-BRCA cohort was selected as an example.

### DETAILED DESCRIPTION OF EMBODIMENTS

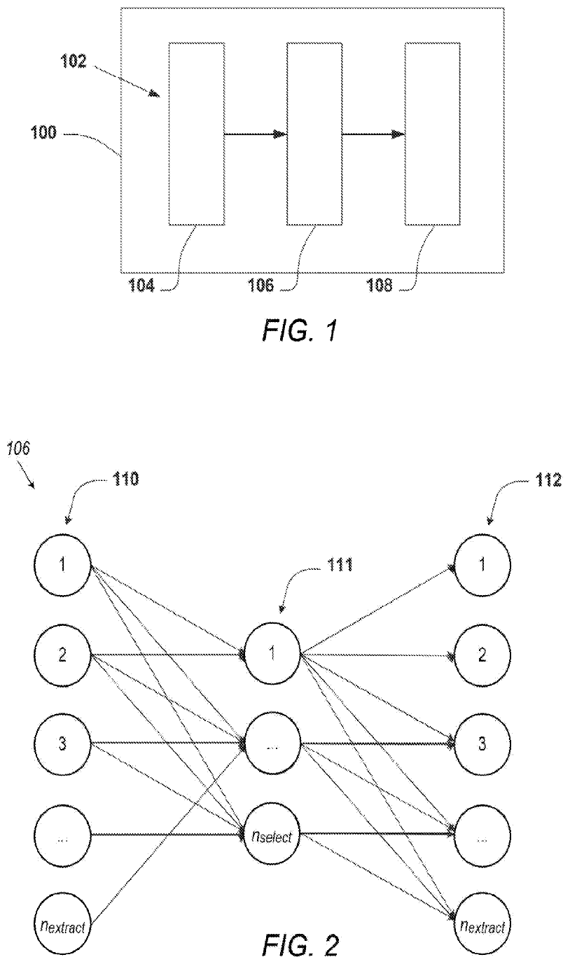

As illustrated in FIG. 1, there is provided system, which is generally indicated at 100, comprising an artificial neural network (ANN) 102, and configured for predicting the expression of each of a plurality of genes, based on a histopathological image input thereto. The ANN 102 comprises a feature extraction module 104, an autoencoder 106, and a multilayer perceptron (MLP) 108.

The feature extraction module 104 is configured to accept the histopathological images, and to extract features therefrom. It may comprise a convolutional neural network (CNN) configured for image recognition, in particular for extracting a number n-extract of features from an image. According to some examples, the CNN comprises a residual neural network. According to a particular example, the CNN implements a ResNet50 architecture, as is well-known in the art. According to some examples, the CNN is pretrained with images from the ImageNet database. According to some examples, the CNN is configured to extract 2,048 features (i.e., according to this example n-extract=2,048).

The autoencoder 106 is configured to select a number n-select of features from those extracted by the feature extraction module 104. The number of selected features n-select may be significantly fewer than the number of extracted features n-extract, for example no more than about a half thereof or no more than about a quarter thereof. The autoencoder 106 may comprise a bottleneck comprising a number n-select of neurons. According to some examples in which the feature extraction module 104 extracts 2,048 features, the autoencoder 106 may be configured to select 512 features, e.g., the bottleneck may comprise 512 neurons (i.e., according to this example n-select=512).

As illustrated in FIG. 2, the autoencoder 106 according to some examples may comprise a first fully connected layer 110 comprising n-extract neurons, a fully connected bottleneck layer 111 comprising n-select neurons, and a second fully connected layer 112 comprising n-extract neurons.

According to some examples, for example as illustrated in FIG. 2, the output neurons of the first fully connected layer 110 constitute the input neurons of the second fully connected layer 112. According to other examples, the output neurons of the first fully connected layer 110 are fully connected to the input neurons of the second fully connected layer 112.

According to some examples, each of the output neurons of one or both of the fully connected layers 110, 112 implements a ReLU (rectified linear unit) activation function.

It will be appreciated that implantation of the autoencoder 106 as described herein may improve performance of the ANN 102. For example, it may exclude noise, avoid overfitting, and/or otherwise reduce computational demands of the ANN 102.

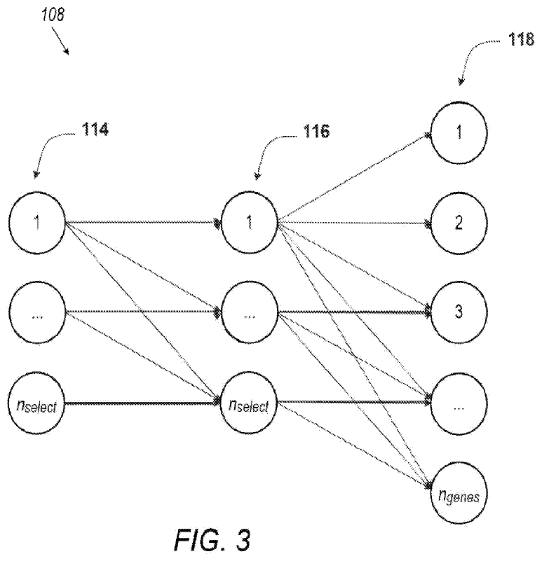

The MLP 108 is connected to the autoencoder 106, and is configured to perform regression using the features compressed by the autoencoder to predict the expression of each of the plurality of genes based on the selected features in the histopathological images. As illustrated in FIG. 3, the MLP 108 may comprise an input layer 114, a hidden layer 116, and an output layer 118. Each of these layers may be a fully connected layer. According to some examples, the neurons of the hidden layer 116 implement a ReLU activation function. According to some examples, the MLP 108 may further comprise a dropout layer (not illustrated) between the hidden layer 116 and the output layer 118.

A skilled artisan would recognize that different types of MLP are available in the art, and that the methods disclosed herein can be implemented using any relevant one. A description of MLPs, their architecture, and learning algorithms thereof can be found, for example in Murtagh F. Neurocomputing 2.5-6 (1991): 183-197 and Popescu, MC. WSEAS Transactions on Circuits and Systems 8.7 (2009): 579-588, the contents of which are incorporated herein in their entirety.

The number of neurons in each of the input and hidden layers 114, 116 may be equal to the number of features compressed by the autoencoder 106, i.e., n-select, and the number of neurons in the output layer 118 may be equal to the number of the plurality of genes, n-genes.

According to some examples, the ANN 102 is configured for simultaneously predicting the expression of each subset of all of the genes of interest. In order to predict the expression of all of the genes of interest, the ANN 102 model is applied to several subsets, which together include all of the genes of interest. Accordingly, the number of neurons in the output layer 118 may be equal to the number of genes of interest (for simplicity, n-genes is used herein to denote the number of genes whose expression is predicted simultaneously by the ANN 102). According to some examples, n-genes=4,096.

According to some examples, the weights from the input layer 114 to the hidden layer 116, representing correlations among the n-genes genes, are shared. According to some examples, the weights from the hidden layer 116 to the output layer 118, representing the predicted expression of each of the genes, are identical.

As mentioned, the ANN 102 is configured for predicting the expression of each of a plurality of genes based on a histopathological image. In particular, it may be configured to predict a gene expression profile of a patient suffering from a pathological condition, for example cancer, based on slide images, e.g., whole slide images (WSIs), of tumor tissue. Accordingly, the ANN 102 is trained with histopathological image data from cancer patients and corresponding gene expression profiles.

Histopathological image data may comprise a plurality of WSIs from an existing catalogue of information collected from cancer patients. According to some examples, the WSIs are retrieved from The Cancer Genome Atlas (TCGA) maintained by the United States National Cancer Institute and National Human Genome Research Institute, and the gene expression profiles comprise corresponding RNAseq gene expression profiles. According to some examples, the histopathological image and gene expression profiles are from patients diagnosed with breast, lung, brain, kidney, colorectal, prostate, gastric, head and neck, cervical, and pancreas cancer.

According to some examples, each of the images are divided into tiles, for example non-overlapping tiles. The terms “tile” and “sub-images” are used herein interchangeably having exactly the same features and limitations. The tiles, or sub-images, may be of any suitable size, e.g., depending on constraints imposed by the ANN 102. According to some examples, each of the tiles may be 512×512 pixels in size.

Tiles, or sub-images, may be excluded from the training data, for example based on quality of the image, insufficient useful data (for example, tiles containing more than a predetermined amount, e.g., 50%, of background may be excluded), etc.

One or more image processing techniques, for example application of color normalization may be applied to minimize effects of staining variation, e.g., heterogeneity and/or batch effects.

According to some examples, the WSIs are formalin-fixed, paraffin-embedded (FFPE) whole slide images.

According to some examples, the tissue is stained with haematoxylin and eosin.

Each of the gene expression profiles is analyzed to select highly expressed genes. In order to reduce the range of gene expression values, as well as to minimize discrepancies in library size between experiments and batches, a normalization may be performed.

Once the image data and gene expression profiles have been prepared, for example as per the above, they are used to train the ANN 102. If necessary, each of the images input into the feature extraction module 102 is resized as necessary. For example, the default input size for a network implementing a ResNet50 architecture is 224×224 pixels. Accordingly, when such an architecture is used, each of the input images, e.g., the tiles, may be resized to be 224×224 pixels.

As mentioned above, the ANN 102 may be configured for simultaneously predicting the expression of each of a subset of all of the genes of interest. According to some examples, each subset of genes comprises genes whose gene expression values were found to be similar when the gene expression profiles were analyzed. This mitigates the risk of the ANN 102 focusing on the most highly expressed genes.

According to some examples, training the ANN may comprise performing a 5×5 nested cross-validation may be performed. For each of the types of cancers, WSIs may be randomly split into five disjoint sets. Each sample set is selected in turn as the held-out test set (20%), while the rest are used for training (80%). Given an outer split, in the inner loop each model is trained five times by further splitting the training set into internal training and validation sets, performing a five-fold cross validation. A bagging technique may be applied, wherein the predictions from the five different models are averaged, providing a final prediction for each gene on the held-out test set. The outer loop may be repeated five times across the five held-out test sets, thereby providing twenty-five trained models.

According to some examples, the Pearson correlation between predicted and actual expression values of each gene across the samples in each test set may be evaluated, taking the mean correlation across all folds, in order to evaluate the model performance.

As mentioned, the training may be performed with a subset of the expressed genes simultaneously, for example using 4,096 genes. The genes used simultaneously for training may be those having similar expression values.

According to some examples, training may be repeated until the average correlation per gene between actual and prediction values of gene expression on the validation set does not improve for a predetermined number of continuing epochs, e.g., 50 epochs, and/or until a predetermined maximum number of epochs are met, e.g., 500 epochs.

According to some examples, an Adam optimizer with mean squared error loss function may be employed, for example in the autoencoder 106 and/or MLP 108. The Adam optimizer may use a learning rate of 10⁻⁴ and mini-batches of 32 image tiles per step A dropout of 0.2 may be used, for example to mitigate the risk of overfitting.

The trained model may be validated by applying the trained ANN 102 to predict gene expressions on one or more independent datasets, e.g., the TransNEO breast cancer cohort, the NCI-brain cohort, etc.

The ANN 102 may be implemented using any suitable tool. According to some examples, it may be implemented using Python (e.g., version 3.7.4), using external libraries including, but not limited to, Numpy (e.g., version 1.18.5), Pandas (e.g., version 1.0.5), Scikit-learn (e.g., version 0.23.1), and/or Matplotlib (e.g., version 3.2.2). Libraries used for image processing (e.g., tile partitioning and/or color normalization) may include, but are not limited to, OpenSlide (e.g., version 1.1.2), OpenCV (e.g., version 4.4.0), PIL (e.g., version 6.1.0), and/or color correct (e.g., version 0.9.1). Feature extraction, feature compression, and regression may be implemented using, e.g., PyTorch (e.g., version 1.7.0). In addition, Scipy (e.g., version 1.5.0) may be used to implement other calculations, for example as is known in the art.

According to some examples, the gene expressions predicted by the ANN 102 may be used to predict the patient's response to a range of targeted therapies and immunotherapies. According to some examples, the predicted gene expressions are used by a transcriptomics-based computational approach to predict the effectiveness of cancer therapies, e.g., by identifying clinically relevant genomic interactions. Such an approach may be provided, e.g., using the ENLIGHT platform provided by Pangea Biomed of Tel Aviv, Israel, and/or the SELECT (synthetic lethality and rescue-mediated precision oncology via the transcriptome) framework developed by Joo Sang Lee, et al. A suitable therapeutic intervention may be selected based on the predicted patient response, and the patient may be treated accordingly.

According to some examples, the respective responses of several patients having a pathological condition may be ranked, e.g., according to the likelihood of success of treatment using a therapeutic intervention, by applying methods described above. The method may be applied, e.g., as part of a clinical trial in order to select subjects with a higher likelihood of having a successful outcome.

According to some examples, the response of a subject to a therapeutic intervention can be monitored by the methods disclosed herein, e.g., histopathological images obtained from said subject can be routinely monitored to prognose the outcome of different therapeutic interventions, thus enabling choosing the intervention with highest chances of success.

It will be appreciated that the type of slide analyzed by the ANN 102 does not need to be the same type used to train it. For example, FFPE slide images may be used to train the ANN 102, but gene expressions may be predicted thereby based on other types of slides, e.g., fresh frozen slides, etc.

According to some examples, the methods disclosed herein comprise utilizing the ENLIGHT or SELECT algorithms for predicting the response to a therapeutic intervention in a patient, based on the gene expression profile of said patient. The ENLIGHT and SELECT algorithms have been described, for example, in Dinstag et al. Med. 4(1):15-30.e8 and in Lee et al. Cell 184(9):2487-2502, a summary of which is included herein.

According to some examples, SELECT drug response prediction algorithm comprises two steps: (1) given a drug, the genetic interactions (GI) engine identifies the clinically relevant GI network of the drug's target genes. A list of initial candidate SL/SR pairs is obtained by analyzing cancer cell line dependencies with RNAi, CRISPR-Cas9, or pharmacological inhibition, based on the principle that SL/SR interactions should decrease/increase tumor cell viability, respectively, when activated. Among these candidate pairs, those that are more likely to be clinically relevant are selected by analyzing a database of tumor samples with associated transcriptomics and survival data, requiring a significant association between the joint inactivation of a putative SL gene pair and better patient survival, and analogously for SR interactions. Finally, among the candidate pairs that remain after these steps, those pairs that are supported by a phylogenetic profiling analysis are selected. When considering combination therapies, a GI network is computed based on the union of all the drug targets. (2) The drug-specific GI network is then used to predict a given patient's response to the drug based on the gene expression profile of the patient's tumor. A matching score, which evaluates the match between patient and treatment, is based on the overall activation state of the genes in the drug target's GI network, reflecting the notion that a tumor would be more susceptible to a drug that induces more active SL interactions and fewer active SR interactions.

The ENLIGHT algorithm, introduces the following adaptations over SELECT: (1) ENLIGHT's GI networks include both SL and SR interactions that are concomitantly identified for each drug, thereby considerably increasing drug coverage relative to SELECT, which utilizes only one type of interaction per network. (2) A depletion test is also used (not present in SELECT), requiring that the joint inactivation of a candidate SL pair is under-represented in tumors (and analogously for SR partners). (3) SELECT has used a Cox proportional hazard test on categorized expression data to identify candidate SL/SR pairs that confer favorable/unfavorable patient survival when the interaction is active. To increase robustness and statistical power, ENLIGHT applies a fully parametric test, based on an exponential survival model, on continuous expression data. (4) ENLIGHT GI networks are considerably larger than those of SELECT to reduce score variation across drugs and indications. (5) To improve the prediction engine, for immunotherapy and other mAbs, which are highly target specific, the ENLIGHT Matching Score (EMS) incorporates the expression of the target as an additional score component.

The ENLIGHT algorithm can be broadly divided into two steps: (i) the GI engine and (ii) the prediction engine.

The GI engine uses 4 statistical tests to identify interacting gene pairs:
in-vitro test. By definition, it is expected that gene A will be more essential when its SL partner gene B is inactive in a cancer cell line. Using a set of input genome-wide shRNA/siRNA/sgRNA screens mined from DepMap, ENLIGHT identifies pairs that show conditional essentiality: Gene A is defined as SL conditionally essential with gene B if its essentiality is significantly higher in cell-lines where gene B is inactive using Wilcoxon rank sum test. Both mRNA expression and SCNA data are used to classify genes as over/under-active. Similarly, gene A is defined as SR-DU/SR-DD conditionally essential with gene B if its essentiality is significantly higher in the samples where gene B is underactive/overactive. An SR-DU/SR-DD interaction between gene A and gene B dictates that a cell can be rescued from cell death caused by the inhibition of gene A by the upregulation/downregulation of gene B respectively.

Depletion test. ENLIGHT requires SL/SR pairs not to display joint activation patterns that are disadvantageous for SL/SR interactions in patient tumors. This requirement is implemented by a depletion test, added as a step to the GI engine. Conceptually, if an SL interaction exists between a pair of genes, we would expect not to observe tumors in which both genes are inactive, since this would have caused tumor cell lethality. Similarly, we would not expect patients with inactivation of a gene to have low/high activation of its SR-DU/SR-DD rescuer, since that would induce tumor cell death. To summarize, in both SL/SR cases we are looking for the statistical absence (or depletion) of a non-favorable joint activation pattern in observational cohorts to support a GI between gene pairs.

Survival test. This test identifies candidate SL/SR pairs that confer favorable/unfavorable patient survival when the interaction is active. When an SL pair is simultaneously inactive in a patient or a cell-line we term it active. Similarly, we term an SR-DU/SR-DD as active when gene A is inactive in conjunction with its SR-DU/SR-DD partner being underactive/overactive. The survival test used in ENLIGHT is a fully parametric test, based on an exponential survival model. Given the omics of a gene pair from a patient cohort, coupled with survival data, we first calculate a covariate value for each patient, reflecting its joint activation state of the gene pair. For a true SL/SR pair, patients in whom the joint activation of the pair is in a disadvantageous state in the tumor, are expected to have better survival, since this should lead to tumor cell death. Hence, the covariate value is positively associated with survival time in these cases. The statistical model of the test follows the common assumption that covariates have a log-linear effect on survival times, and the coefficient of the SL/SR covariate reflects whether a putative interaction confers a significant effect on patient survival. The model also controls for patient age, gender and stage as confounding factors. If data is missing for any of these three attributes in a patient, we set it to the mean of all other patients (or the majority in the case of gender).

Phylogenetic test. The last test identifies SL/SR pairs with high similarity between their phylogenetic profiles, following observations that interacting genes were found to be conserved across different species and following the analysis of Lee et al. This is done by calculating the Euclidean distance between the genetic similarity profiles of two genes A and B across 86 species, while taking into account the baseline phylogenetic distance between the species (adopting the method of Tabach et al.).

In order to build a GI network around specific drug target/s, we start by performing the above 4 tests for all putative SL/SR-DU/SR-DD pairs between the targets of the drug and all other genes in the genome. Then, we sequentially filter out non-significant pairs for each test, starting from all pairs and all interaction types, so that only pairs that pass all 4 tests are kept. We follow Lee et al. for setting the statistical significance thresholds. Finally, we rank the remaining interactions according to the survival test as it best reflects the clinical impact of the interactions, and use the top K interactions to build the GI network. In this study we tested K=25, 50, 100, 150 and 200 on the tuning sets and selected K=100 as it achieved the best PPV. For immunotherapies, ENLIGHT uses the same GI networks used in SELECT (K=10).

Prediction engine. The prediction engine predicts the response to a given drug or a given combination of drugs with known drug targets based on quantitative RNA data (microarray or RNAseq). The prediction engine works as follows:

First, a GI network surrounding all drug targets is generated based on the GI engine. Next, the RNA data of the cohort is rank normalized to values in [0,1] twice: gene-wise and patient-wise. Gene-wise normalized values are used to identify gene activation states of SL/SR partners across comparable samples of the same tissue. Patient-wise normalized values are used to determine whether the drug targets are expressed to a minimal degree in a patient for the drug to have an effect. The ENLIGHT Matching Score (EMS) is defined as the fraction of SL/SR interactions that are in an advantageous predisposition for drug admission. That is, a drug that inhibits gene A is expected to work better in patients for whom an SL/SR-DU B of A is underexpressed, or for whom an SR-DD partner B of A is overexpressed. Thus, the fraction of under/over expressed partners in advantageous states (with respect to the interaction type) is expected to be positively associated with response. A gene is determined to be underexpressed if its normalized expression is equal to or below ⅓ (i.e. is in the bottom tertile across samples in the same dataset), or overexpressed if its normalized expression is equal to or above ⅔ (i.e. is in the top tertile across samples in the same dataset). In addition, we zero the EMS of a patient if its mean patient-wise normalized expression of drug targets is below or equal to the 30th percentile across genes. This has been motivated by the notion that an antagonist drug will not be effective when its target genes are underexpressed. Finally, for treatments that are highly target specific, namely ICB and other mAbs, the EMS incorporates the target expression, since the drug is expected to be more effective when the target expression is higher. Specifically, the EMS is a geometric mean of the network-based score and a logistic function of the target expression.

A skilled artisan would appreciate that the ENLIGHT or the SELECT algorithms can be readily adapted to use gene expression profiles predicted from histopathological images. Disclosed herein is a method for identifying a pair of genes comprising a synthetic lethality (SL), synthetic rescue (SR), or synthetic dosage lethality (SDL) interaction, wherein said pair of genes is identified by obtaining gene expression profiles from histopathological slides, and using said gene expression profiles as an input for the ENLIGHT or SELECT algorithms, thereby identifying pair of genes comprising SL, SR, or SDL.

Further, ENLIGHT or SELECT algorithms adapted to use gene expression profiles predicted from histopathological images, can be used for identifying a drug target candidate. In some embodiments, said method comprises identifying genes comprising SL, SR, or SDL interactions using an ENLIGHT or a SELECT algorithms; wherein said ENLIGHT or said SELECT algorithm use gene expression profiles obtained from histopathological images.

Similarly, ENLIGHT or SELECT algorithms adapted to use gene expression profiles predicted from histopathological images, can be used to find novel therapeutic combinations. In some embodiments, disclosed herein is a method of predicting the efficiency of novel therapeutic combinations using ENLIGHT or a SELECT algorithm; wherein said ENLIGHT or said SELECT algorithms use gene expression profiles obtained from histopathological images.

### EXAMPLES

### Example 1—Synthetic Lethality-Based Prediction of Cancer Treatment Response from Histopathology Images

Advances in artificial intelligence have paved the way for leveraging hematoxylin and eosin (H&E)-stained tumor slides for precision oncology. We present ENLIGHT-DeepPT, an approach for predicting response to multiple targeted and immunotherapies from H&E-slides. In difference from existing approaches that aim to predict treatment response directly from the slides, ENLIGHT-DeepPT is an indirect two-step approach consisting of (1) DeepPT, a new deep-learning framework that predicts genome-wide tumor mRNA expression from slides, and (2) ENLIGHT, which predicts response based on the DeepPT inferred expression values. DeepPT successfully predicts transcriptomics in all 16 TCGA cohorts tested and generalizes well to two independent datasets. Importantly, ENLIGHT-DeepPT successfully predicts true responders in five independent patients' cohorts involving four different treatments spanning six cancer types with an overall odds ratio of 2.44, increasing the baseline response rate by 43.47% among predicted responders, without the need for any treatment data for training. Furthermore, its prediction accuracy on these datasets is comparable to a supervised approach predicting the response directly from the images, trained and tested on the same cohort in cross validation. Its future application could provide clinicians with rapid treatment recommendations to an array of different therapies and importantly, may contribute to advancing precision oncology in developing countries.

### INTRODUCTION

Histopathology has long been considered the gold standard of clinical diagnosis and prognosis in cancer. In recent years, molecular markers including tumor gene expression have proven increasingly valuable for enhancing diagnosis and precision oncology. Digital histopathology promises to combine these complementary sources of information using machine learning, artificial intelligence and big data. Key advances are already underway, as whole slide images (WSI) of tissue stained with haematoxylin and eosin (H&E) have been used to computationally diagnose tumors, classify cancer types, distinguish tumors with low or high mutation burden, identify genetic mutations, predict patient survival, detect DNA methylation patterns and mitoses, and quantify tumor immune infiltration. Previous work has already impressively unravelled the potential of harnessing next-generation digital pathology to predict response to therapies directly from images. In these direct supervised learning approaches, predicting response to therapy directly from the WSI requires large datasets consisting of matched imaging and response data. As such, they require a specific cohort for each drug/indication treatment that is to be predicted. However, the availability of such data on a large scale is still fairly limited, restricting the applicability of this approach and raising concerns about the generalizability of supervised predictors to other cohorts.

To overcome this challenge, we turned to develop and study a generic methodology for generating WSI-based predictors of patients' response for a broad range of cancer types and therapies, which does not require matched WSI and response datasets for training. To accomplish this, we have taken an indirect two-step approach: First, we developed DeepPT (Deep Pathology for Transcriptomics), a novel deep-learning framework for imputing (predicting) gene expression from H&E slides, which extends upon previous valuable work on this topic. The DeepPT models are cancer type-specific and are built by training on matched WSI and expression data from the TCGA. Second, given gene expression values predicted by these models for a new patient, we apply our previously published approach, ENLIGHT (Dinstag et al. Med. 4(1):15-30.e8), originally developed to predict patient response from measured tumor transcriptomics, to predict response from the DeepPT imputed transcriptomics.

We proceed to provide an overview of DeepPT's architecture and a brief recap of ENLIGHT's workings, the study design and the cohorts analysed. We then describe the results obtained, in each of the two steps of ENLIGHT-DeepPT. First, we study the ability to predict tumor expression, showing the performance of the trained DeepPT models in predicting the gene bulk expression in 16 TCGA cohorts and in two independent, unseen cohorts. Second, we analyze five independent clinical trial datasets of patients with different cancer types that were treated with various targeted and immune therapies. Critically, those are test cohorts, on which DeepPT was never trained. We show that ENLIGHT, adhering to the parameters used in its original publication (Dinstag et al. Med. 4(1):15-30.e8) without any adaptation, can successfully predict the true responders from the expression values imputed by DeepPT, using only H&E images. We then compare its prediction accuracy to that of a direct approach that predicts the response directly from the images. Overall, our results show that combining digital pathology with an expression-based response prediction approach offers a promising new way to provide clinicians with almost immediate treatment recommendations that may help guide patients' treatment until more information arrives from multi-omics biomarker screens.

### METHODS

### Data Collection

The datasets in this study were collected from three resources: TCGA, TransNEO, and Laboratory of Pathology at the NCI. TCGA histological images and their corresponding gene expression profiles were downloaded from GDC (https://portal.gdc.cancer.gov). Only diagnostic slides from primary tumors were selected, making a total of 6,269 formalin-fixed paraffin-embedded (FFPE) slides from 5,528 patients with breast cancer (1,106 slides; 1,043 patients), lung cancer (1,018 slides; 927 patients), brain cancer (1,015 slides; 574 patients), kidney cancer (859 slides; 836 patients), colorectal (514 slides; 510 patients), prostate (438 slides; 392 patients), gastric (433 slides; 410 patients), head & neck (430 slides; 409 patients), cervical (261 slides; 252 patients), pancreatic cancer (195 slides; 175 patients).

The TransNEO-Breast dataset consists of fresh frozen slides and their corresponding gene expression profiles from 160 breast cancer patients. Full details of the RNA library preparation and sequencing protocols, as well as digitization of slides have been previously described.

The NCI-Brain histopathological images and their corresponding gene expression profiles were obtained from archives of the Laboratory of Pathology at the NCI, and consisted of 226 cases comprising a variety of CNS tumors, including both common and rare tumor entities. All cases were subject to methylation profiling to evaluate the diagnosis, as well as RNA-sequencing.

### Histopathology Image Processing

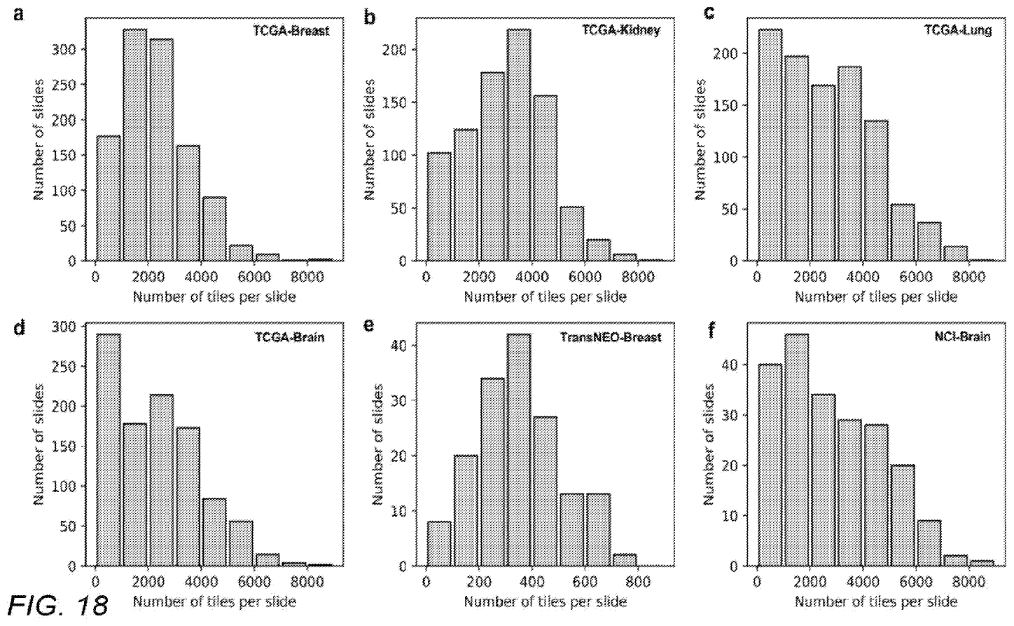

We first used Sobel edge detection to identify areas containing tissue within each slide. Because the WSI are too large (from 10,000 to 100,00 pixels in each dimension) to feed directly into the deep neural networks, we partitioned the WSI at 20× magnification into non-overlapping tiles of 512×512 RGB pixels. Tiles containing more than half of the pixels with a weighted gradient magnitude smaller than a certain threshold (varying from 10 to 20, depending on image quality) were removed. Depending on the size of slides, the number of tiles per slide in the TCGA cohort varied from 100 to 8,000 (FIG. 18a-d). In contrast, TransNEO slides are much smaller, resulting in 100 to 1,000 tiles per slide (FIG. 18e). To minimize staining variation (heterogeneity and batch effects), color normalization was applied to the selected tiles.

### Gene Expression Processing

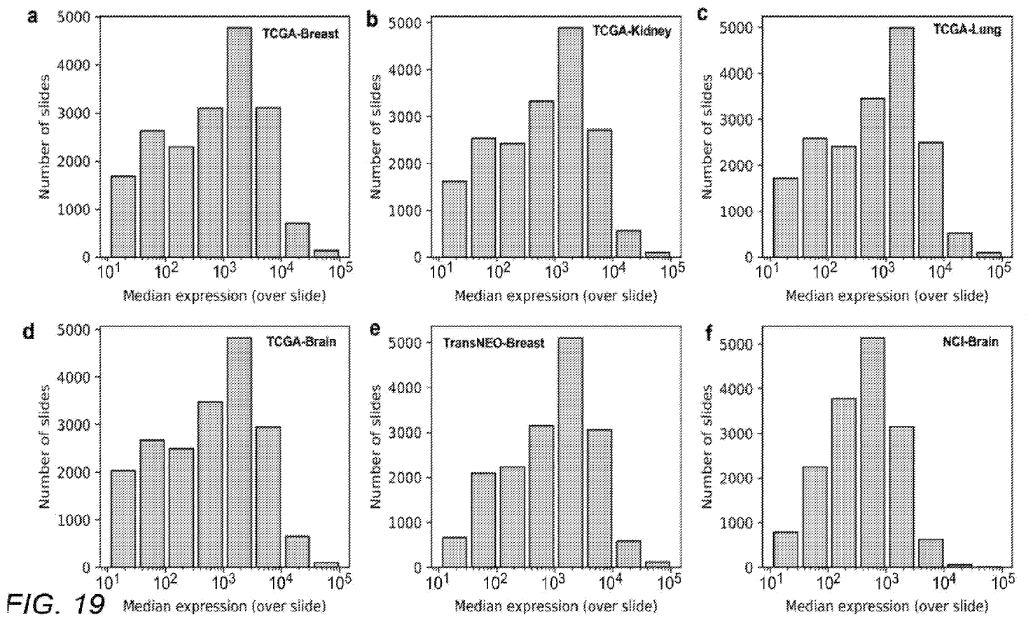

Gene expression profiles were extracted from read counts files which contains approximately 60,000 gene identifiers. A subset of highly expressed genes was identified using edgeR, resulting in roughly 18,000 genes for each cancer type. The median expression over samples of each gene varied from 10 to 10,000 across for every dataset (FIG. 19). To reduce the range of gene expression values, and to minimize discrepancies in library size between experiments and batches, a normalization was performed as described in our previous work.

### Model Architecture

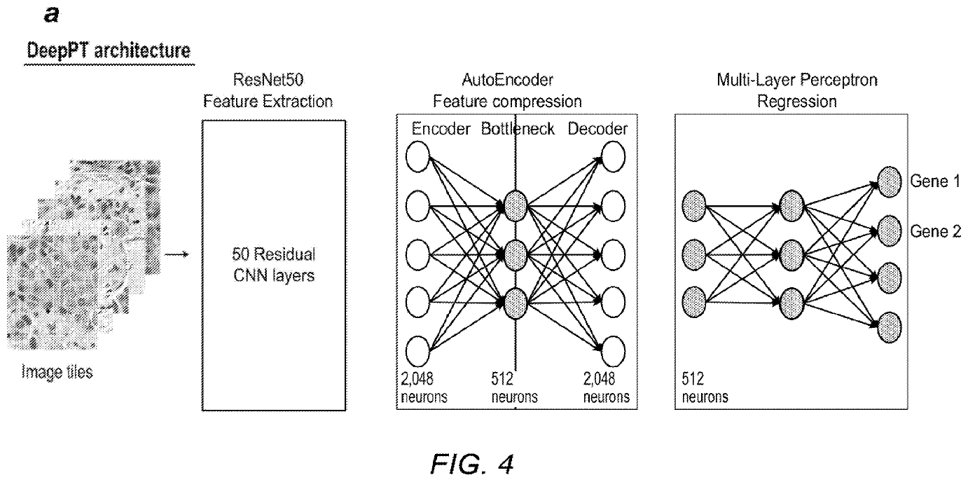

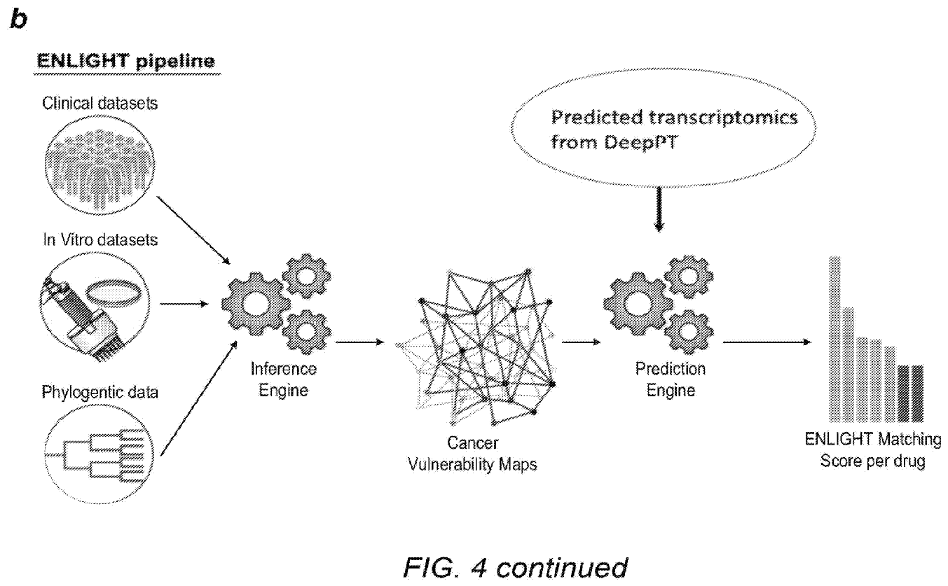

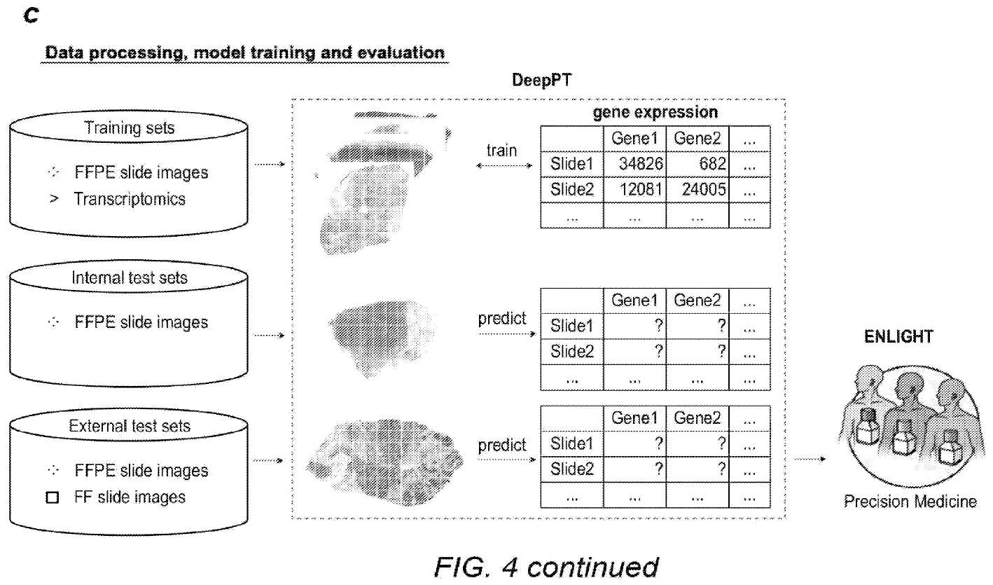

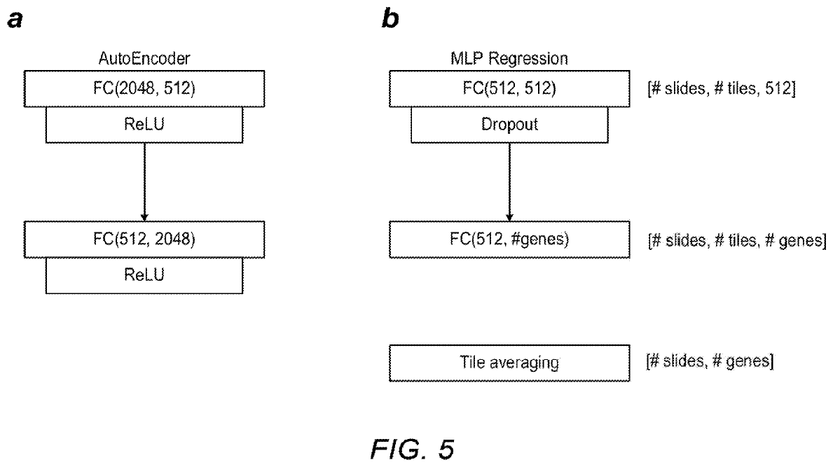

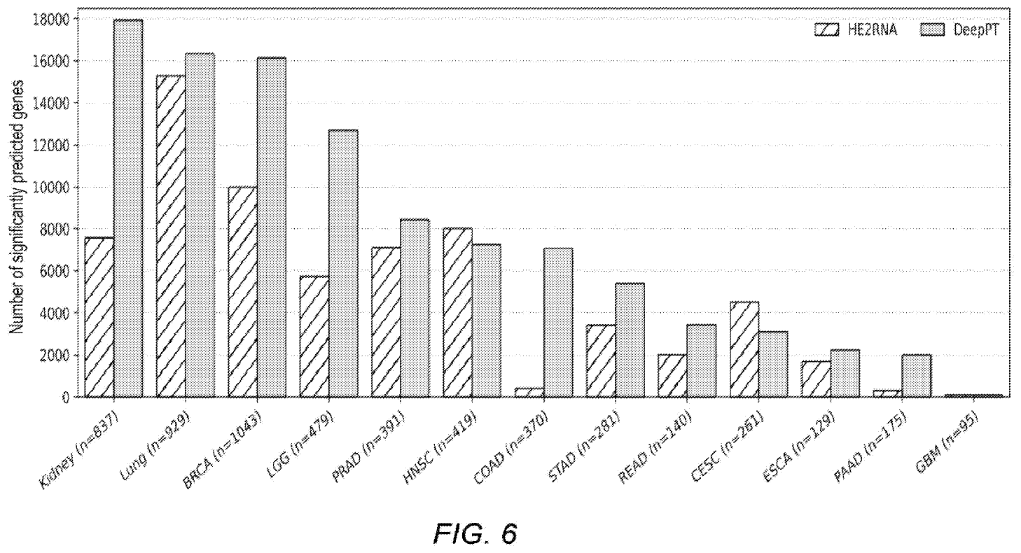

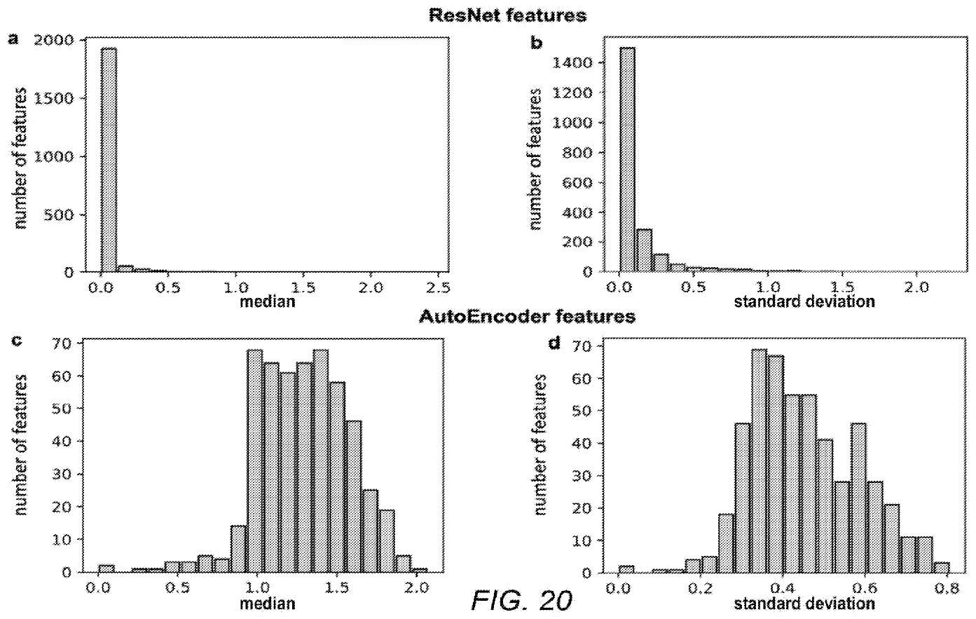

Our model architecture was composed of three main units (FIG. 4 and FIG. 5).
(1) Feature extraction: The pre-trained ResNet50 CNN model with 14 million natural images from the ImageNet database was used to extract features from image tiles. Before feeding these tiles into the ResNet50 unit, the image tiles were resized to 224×224 pixels to match the standard input size for the convolutional neural network. Through the feature extraction process, each input tile is represented by a vector of 2,048 derived features.
(2) Feature compression: We applied an autoencoder, which consists of a bottleneck of 512 neurons, to reduce the number of features from 2,048 to 512. This helps to exclude noise, to avoid overfitting, and finally to reduce the computational demands. As shown in FIG. 20, a large number of ResNet features are constantly zero (FIG. 20 upper panels). This data sparsity is considerably reduced in the autoencoder features (FIG. 20, lower panels).
(3) Multi-Layer Perceptron (MLP) regression: The purpose of this component is to build a predictive model linking the aforementioned auto-encoded features to whole-genome gene expression. The model consists of three layers: (1) an input layer with 512 nodes, reflecting the size of the auto-encoded vector; (2) a hidden layer whose size depends on the number of genes under shared consideration; and (3) an output layer with one node per gene. The rationale behind this architecture is to leverage similarity among the genes under shared consideration, as captured by the weights connecting the input layer to the hidden layer. The weights connecting the hidden layer to the output layer model the subsequent relationship between the hidden layer and each individual gene. This follows the philosophy of multi-task learning. If the prediction of each gene's expression level represents a single task, then our strategy is to first group these tasks for shared learning, followed by optimization of each individual task. In our default whole-genome approach, we bin genes into groups of 4,096 whose median expression levels are similar, and we use 512 hidden nodes. Because the training data is comprised of gene expression at the slide level (i.e. bulk gene expression, as opposed to at spatial resolution), we average our per-tile predictions to obtain bulk values at the slide level.

### Model Training and Evaluation

We trained and evaluated each cancer type independently. To evaluate our model performance, we applied 5×5 nested cross-validation. For each outer loop, we split the entire patients (of each cohort) into training (80%) and held-out test (20%) set. We further split the training set into internal training and evaluation set, according five-fold cross validation. The models were trained and evaluated independently with the different pairs of training/validation sets. Averaging the predictions from the five different models represents our final prediction for each single gene on each held-out test set. We repeated this procedure five times across the five held-out test sets, making a total of 25 trained models. These models trained with TCGA cohorts were used to predict the expression of each gene in a given external cohort by computing the mean over the predicted values of all models. Because each patient can have more than one slide, we average the slide-level predictions to obtain patient-level predictions.

As noted in the Model Architecture section, tranches of genes with similar median expression levels were grouped for simultaneous training and evaluation. This was done to optimize model performance and model efficiency, and contrasts approaches in the literature that either train on each gene separately or on all genes together. Each training round was stopped at a maximum of 500 epochs, or sooner if the average correlation per gene between actual and prediction values of gene expression on the validation set did not improve for 50 continuous epochs. The Adam optimizer with mean squared error loss function was employed in both auto-encoder and MLP models. A learning rate of 10⁻⁴ and a minibatches of 32 image tiles per step were used for both the auto-encoder model and MLP regression model. To avoid overfitting, a dropout of 0.2 was also used.

### Implementation Details

All analysis in this study was performed in Python 3.7.4 and R 4.1.0 with the libraries including Numpy 1.18.5, Pandas 1.0.5, Scikit-learn 0.23.1, Matplotlib 3.2.2, and edgeR 3.28.0. Image processing including tile partitioning and color normalization was conducted with OpenSlide 1.1.2, OpenCV 4.4.0, PIL 6.1.0. The histopathology feature extraction, the feature compression (autoencoder unit) and MLP regression parts were implemented using PyTorch 1.7.0. Pearson correlation was calculated using Scipy 1.5.0. Differential gene expression analysis was performed with edgeR 3.28.0.

### RESULTS

### The Computational Pipeline

DeepPT is trained on formalin-fixed, paraffin-embedded (FFPE) whole slide images and their corresponding gene expression profiles from TCGA patient samples. The model obtained is then used to predict gene expression from both internal held-out and external datasets. In difference from previous studies aimed at predicting gene expression from WSI, which have focused on fine tuning the last layer of a pre-trained convolutional neural networks (CNN), DeepPT is composed of three main components (FIG. 4A): a CNN model for feature extraction, an auto-encoder for feature compression, and a multiple-layer perceptron (MLP) for the final regression. Rather than training a model for each gene separately or for all genes together as was done in previous studies, we trained simultaneously on tranches of genes with similar median gene expression values, allowing shared signals to be leveraged while preventing model from focusing on only the most highly expressed genes.

The predicted expression then serves as input to ENLIGHT, which is a transcriptomics-based approach that predicts individual responses to a wide range of targeted and immunotherapies based on gene expression data measured from the tumor biopsy (FIG. 4B). ENLIGHT aims to advance and extend the scope of SELECT, two recent approaches that rely on analysis of functional genetic interactions (GI) around the target genes of the chosen therapy. Specifically, two broad types of interactions are considered: Synthetic Lethality (SL), whereby the simultaneous loss of two non-essential genes is lethal to the cell, and Synthetic Rescue (SR), whereby the loss of an essential gene can be compensated for through the over-or under-expression of a second gene (its “rescuer” gene). ENLIGHT's drug response prediction pipeline comprises two steps (FIG. 4B): (i) Given a drug, the inference engine identifies the clinically relevant genetic interaction partners of the drug's target gene(s). The inference engine first identifies a list of initial candidate SL/SR by analysing cancer cell line dependencies based on the principle that SL/SR interactions should decrease/increase tumor cell viability, respectively, when ‘activated’ (e.g., in the SL case, viability is decreased when both genes are under-expressed). It then selects those pairs that are more likely to be clinically relevant by analysing a database of tumor samples with associated transcriptomics and survival data, requiring a significant association between the joint inactivation of target and partner genes and better patient survival for SL interactions, and analogously for SR interactions. (ii) The drug-specific GI partners are then used to predict a given patient's response to the drug based on the gene expression profile of the patient's tumor. The ENLIGHT Matching Score (EMS), which evaluates the match between patient and treatment, is based on the overall activation state of the set of GI partner genes of the drug targets, reflecting the notion that a tumor would be more susceptible to a drug that induces more active SL interactions and fewer active SR interactions.

### Study Design and Patient Cohorts

The workflow describing the computational analysis is depicted in FIG. 4C. First, to build cancer type specific DeepPT models, we collected FFPE WSI together with matched RNAseq gene expression profiles for 16 cancer types from TCGA, composing 10 broad classes (some broad cancer indications include a few types, as per the original TCGA nomenclature): breast (BRCA), lung (LUAD and LUSC), brain (LGG and GBM), kidney (KIRC, KIRP and KICH), colorectal (COAD and READ), prostate (PRAD), gastric (STAD and ESCA), head and neck (HNSC), cervical (CESC), and pancreas (PAAD). These were chosen as they are major cancer types and/or have corresponding external datasets for evaluating purposes. Low quality slides (heavily marked, blurry, damaged, or too small) were excluded, resulting in 6,269 slides from 5,528 patients (Table 1). Each cancer type was processed, trained and evaluated separately. We performed a five-fold cross-validation to evaluate the model performance: In each loop, the patients were randomly split into five disjoint sets. Each of these sets was selected in turn as the held-out test set (20%), while the rest were used for training (80%). Note that the test set remained completely unseen during the model training, and the splits were performed at the patient level so that slides from the same patients are assigned to the same set to avoid information leakage between test and training sets. For further validation, we applied the trained models (with the TCGA cohort) to predict gene expression on two independent external datasets, including the TransNEO breast cancer cohort (TransNEO-Breast) consisting of 160 FF slides, and a new unpublished brain cancer cohort (NCI-Brain) consisting of 226 FFPE slides, both also containing matched expression data. Our final goal is to use the inferred gene expression as input to ENLIGHT based predictions of patients treatment response. To this end, we further applied the DeepPT models to predict gene expression in five clinical trial datasets, as described in the next section.

### Prediction of Gene Expression from Histopathology Images

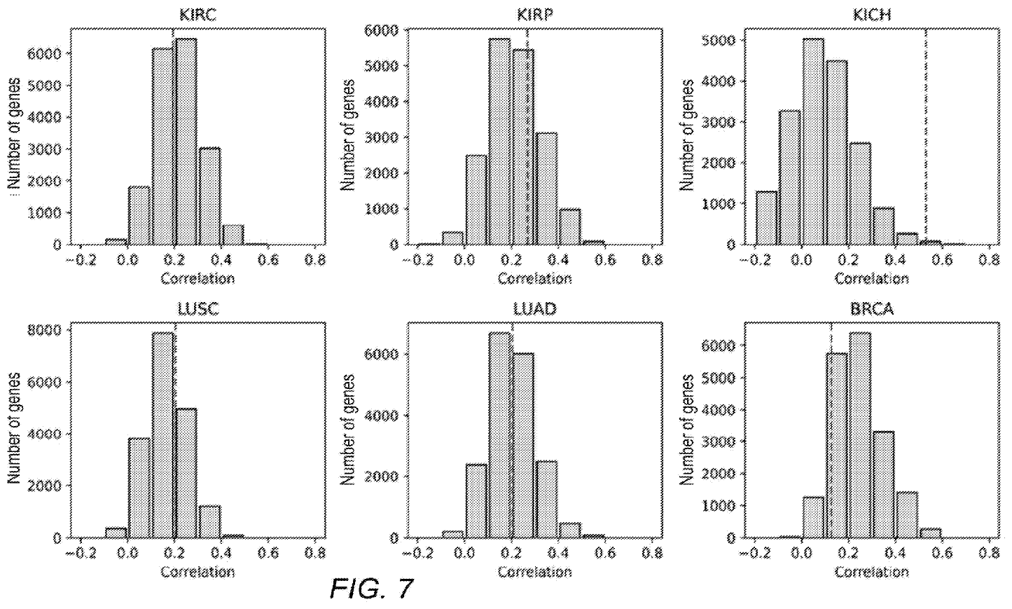

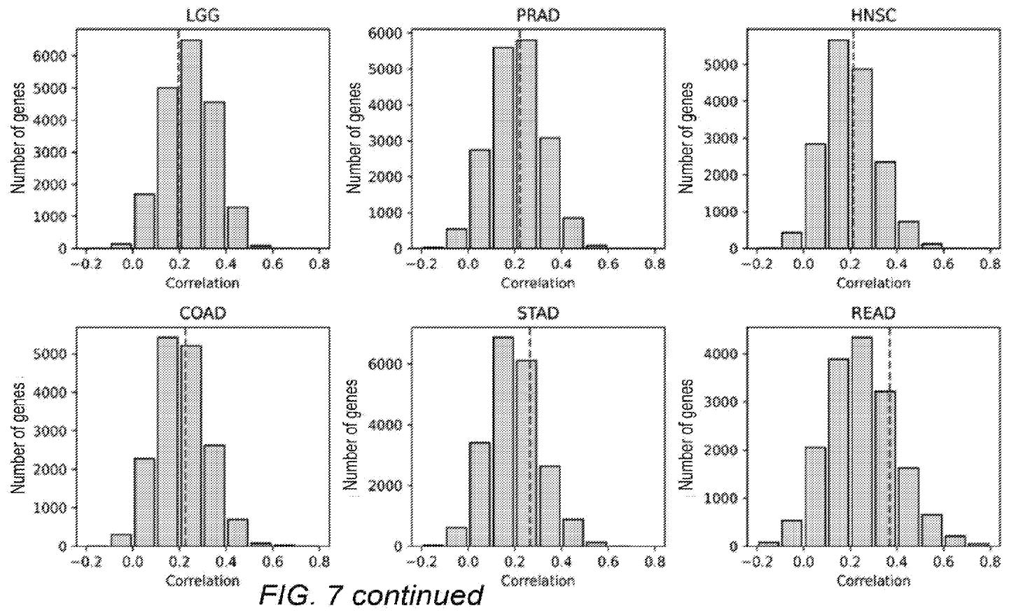

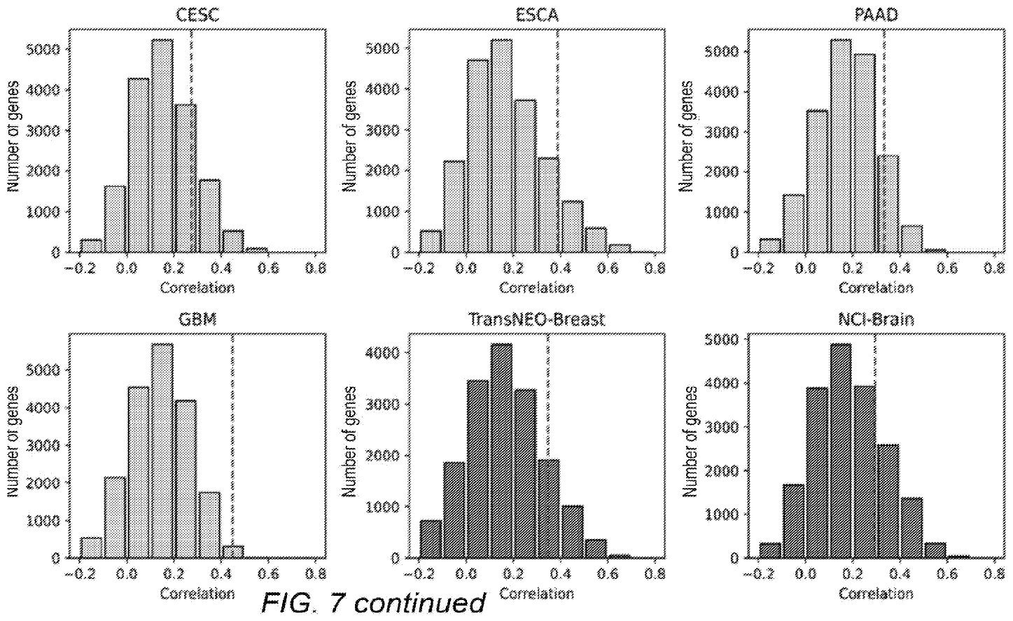

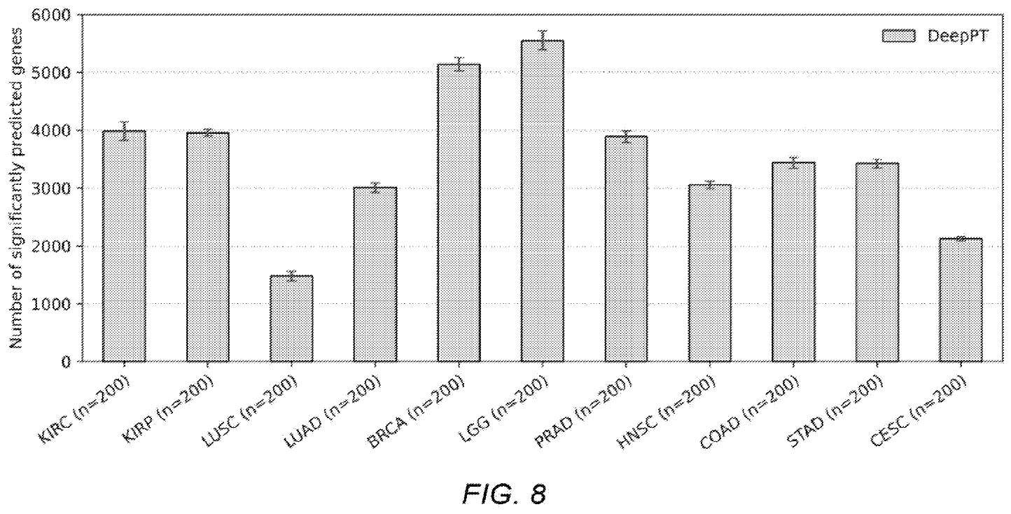

As illustrated in FIG. 4, we constructed models predicting normalized gene expression profiles from their corresponding histopathology images for each of 10 broad TCGA cancer classes. We then applied the trained models to predict gene expression of internal held-out test sets in each of the cancer types, using five-fold cross validation. In most cancer types, thousands of genes were significantly predicted, with Holm-Sidak corrected p-values<0.05. These results outperform the recently published state-of-the-art expression prediction approach, HE2RNA by Schmauch et al (Nat Commun. 2020; 11:3877), in most cohorts considered in this study. To further evaluate model performance, we estimated the Pearson correlation (R) between predicted and actual expression values of each gene across the test dataset samples. In most cancer types, thousands of genes had a correlation above 0.4 (FIG. 7). For breast cancer, for instance, DeepPT predicted 1,812 genes with mean correlation greater than 0.4, more than doubling the number of genes predicted at that correlation level reported by HE2RNA, which was 786 genes, further testifying to the increased accuracy of DeepPT.

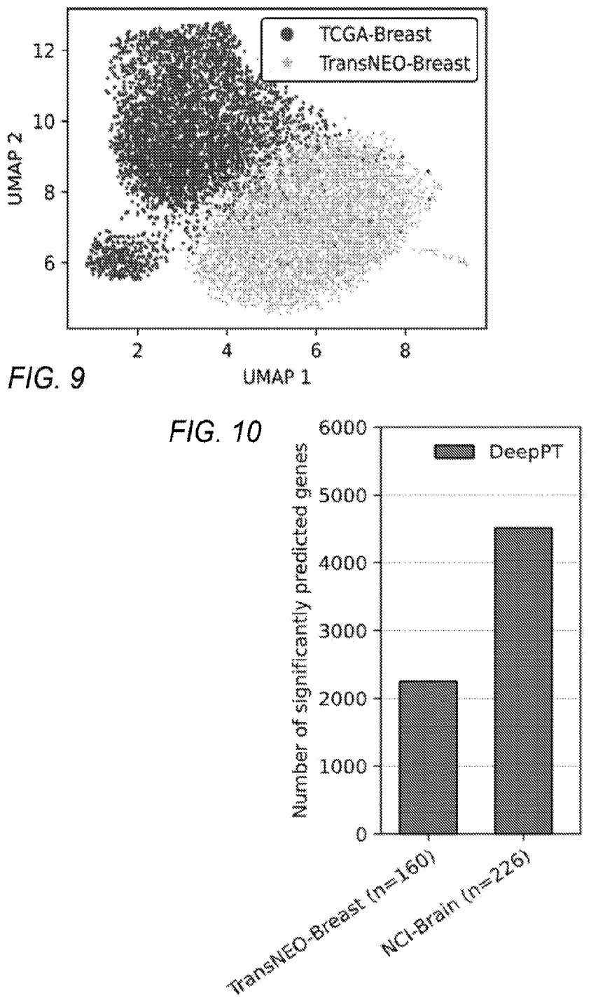

For external validation, we tested the prediction ability of DeepPT on two unseen independent datasets available to us, which contained matched tumor WSIs and gene expression. We first applied the DeepPT model constructed using the TCGA-Breast cancer dataset to predict gene expression from corresponding H&E slides of the TransNEO breast cancer cohort (n=160). Notably, the two datasets were generated independently at different facilities, with two different preparation methods (TCGA slides are FFPE while TransNEO slides are FF), so the histological features extracted from these two datasets are quite distinct (FIG. 9). Despite these differences, without any further training or tuning, we found 2,248 genes that were significantly predicted (FIG. 10). Similarly, we applied the DeepPT model trained on TCGA-Brain samples to predict gene expression from new unpublished NCI-Brain slides (n=226) and found that 4,510 genes were significantly predicted (FIG. 10). This testifies to the considerable predictive power and generalizability of DeepPT.

### Genes Reliably Predicted by DeepPT are Enriched for Cancer Hallmarks

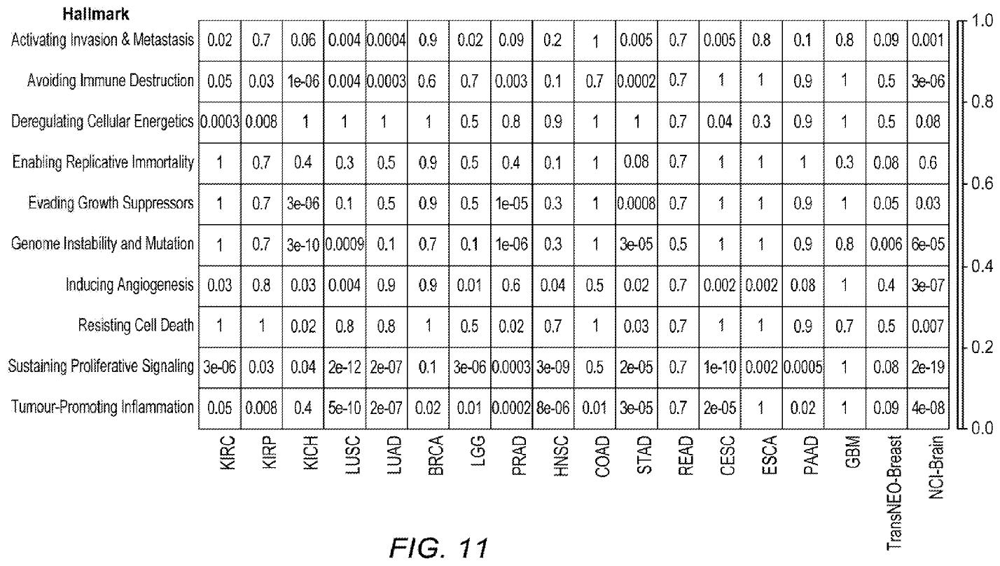

We next explored whether genes that are reliably predicted by DeepPT, i.e. those that are significantly correlated between predicted and measured expressions, have biological relevance to cancer. To this end, we carried out a pathway enrichment analysis (PEA) focused on cancer hallmarks. Specifically, we looked for enrichment among 10 cancer hallmarks described by Hanahan and Weinberg and for which detailed gene sets were given by Iorio et al. FIG. 11 summarizes the PEA results for all TCGA subtypes and the two external cohorts. Interestingly, we observed a strong enrichment for immune processes across the vast majority of cancer types (bottom row, “Tumor-promoting inflammation”), testifying to the important role of immune processes in shaping tumor morphology, as reflected by the slide images. Other enriched hallmarks include the cell cycle (“Sustaining proliferative signaling”), “Avoiding immune destruction” and “Activating invasion and metastasis”. Notably, these results are consistent and even stronger for the external datasets (TransNEO-Breast and NCI-Brain). They further testify that DeepPT can faithfully reconstruct key elements in cell expression related to cancer.

### Predicting Treatment Response from DeepPT-Imputed Gene Expression

As described earlier, the goal of ENLIGHT-DeepPT is to predict patients' response from WSI without any training on the evaluation cohort. Given a previously unseen tumor slide image, we first apply the pre-trained, cancer-type specific DeepPT model to predict the tumor transcriptomics. Second, based on this predicted gene expression, we apply our published precision oncology algorithm, ENLIGHT, to predict the patient's response.

We tested the ability of ENLIGHT-DeepPT to accurately predict patient response in five clinical cohorts, treated with various targeted and immunotherapies, for which patient slides and response data were available. Those include two HER2+ breast cancer patient cohorts treated with chemotherapy plus Trastuzumab, a BRCA+ pancreatic cancer cohort treated with PARP inhibitors (Olaparib or Veliparib), a mixed indication cohort of Lung, Cervical and Head & Neck patients treated with Bintrafusp alfa, a bi-specific antibody that targets TGFB and PDL1, and finally, an ALK+ NSCLC cohort treated with ALK inhibitors (Alectinib or Crizotinib). For each dataset, the response definition was determined by the clinicians running the respective trial (Table 2 for more details). As ENLIGHT does not predict response to chemotherapies, only the targeted agents were considered for response prediction.

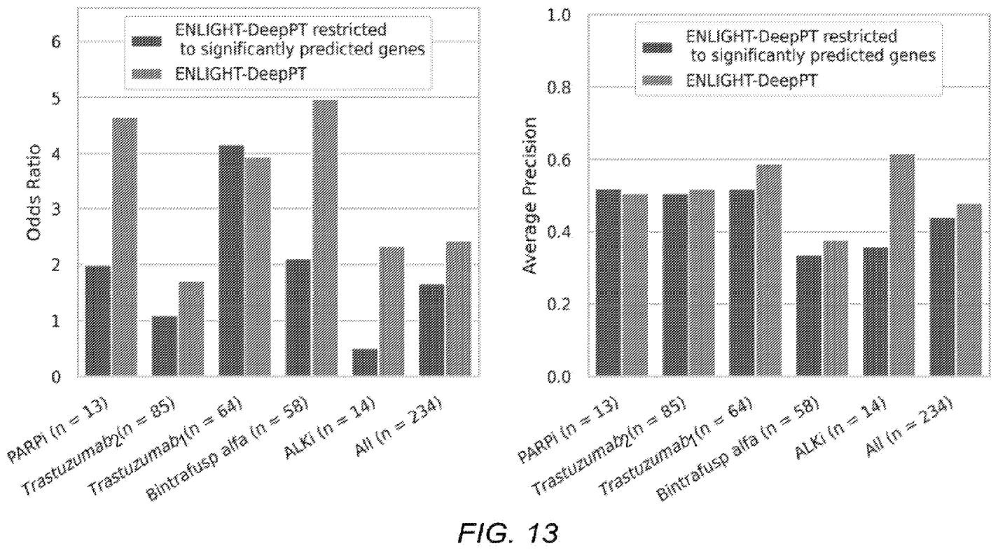

For each cohort, we used the DeepPT model previously trained on the appropriate TCGA cohort, without any changes and with no further training, to predict the gene expression values from the H&E slide of each patient's pre-treated tumor. We then applied ENLIGHT to these predicted gene expression values to produce ENLIGHT Matching Scores (EMS) based on the genetic interaction network of the given drug, as was originally published in (Dinstag et al. Med. 4(1):15-30.e8). Importantly, we do not restrict the GI network to include only genes with strong correlations between the actual and predicted expression values; This is done as ENLIGHT considers the combined effect of a large set of genes, averaging out noise arising from individual gene expression prediction. Notably, restricting ENLIGHT's GI networks to include only significantly predicted genes does not improve results (FIG. 13).

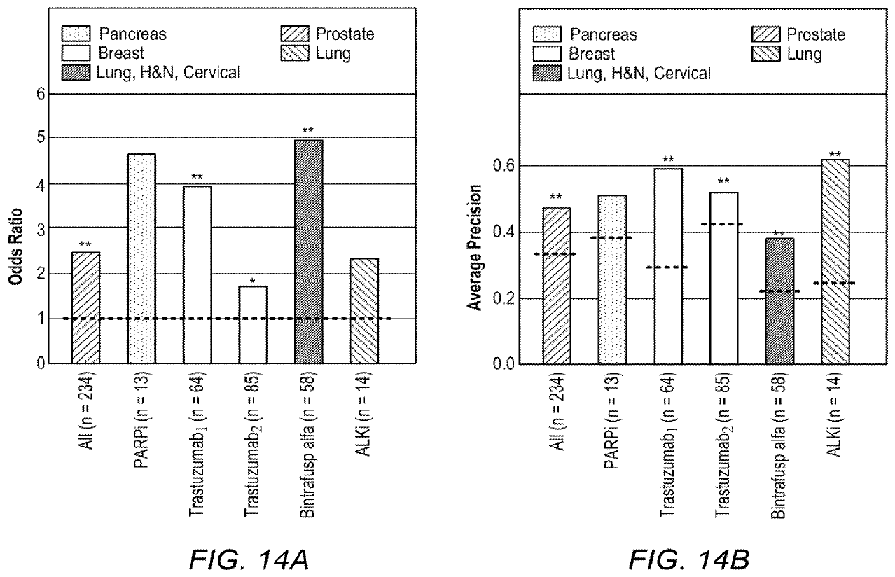

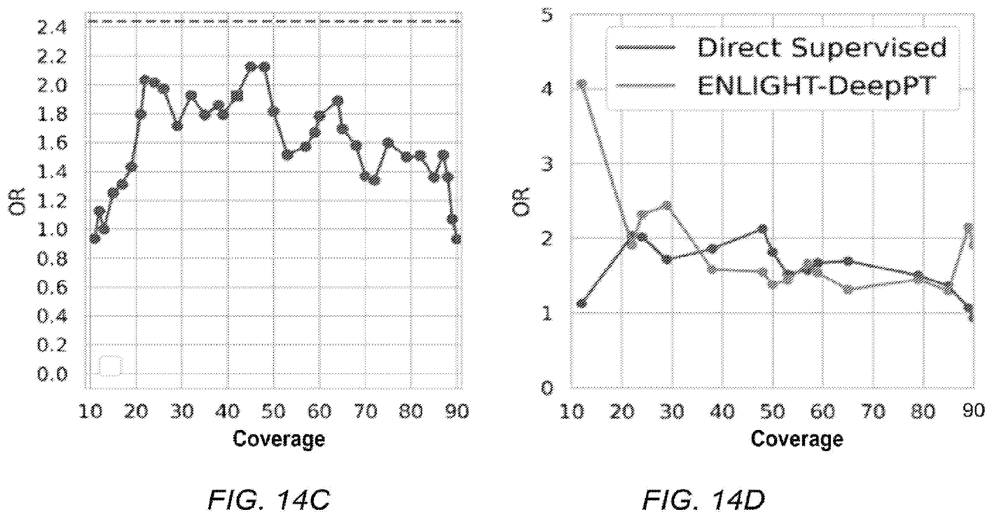

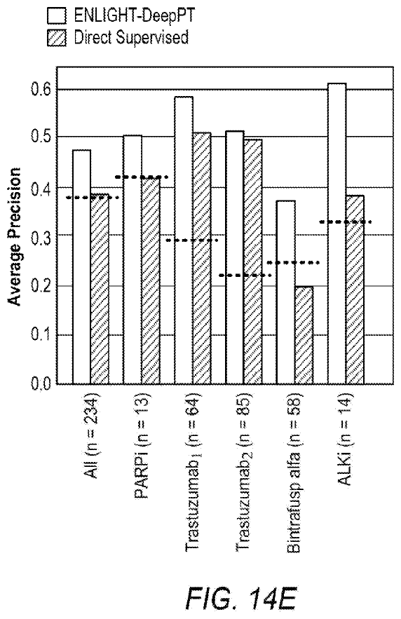

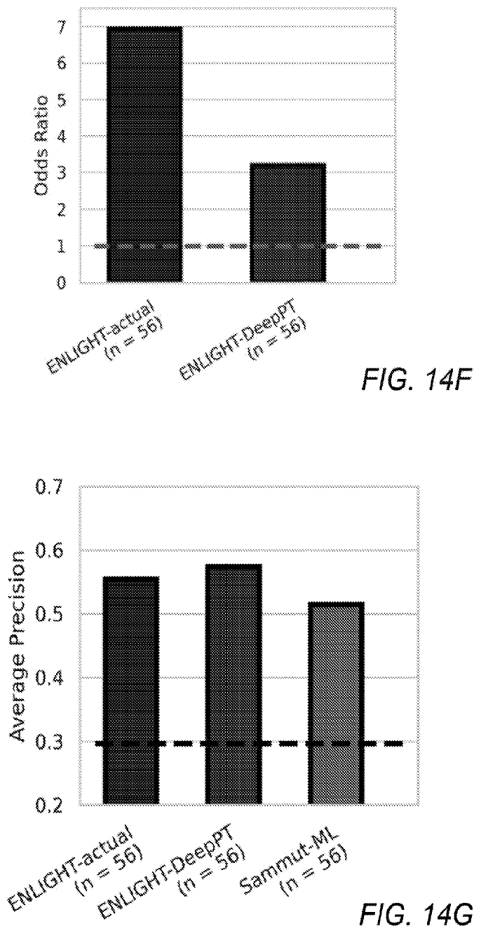

The prediction accuracy of ENLIGHT-DeepPT in each of these five datasets individually and in aggregate is shown in FIG. 14. Since the ENLIGHT-DeepPT workflow is designed with clinical applications in mind, we focus our assessment of its predictive power on measures that have direct clinical importance, including both the odds ratio (OR) of response and the average precision. The OR denotes the ratio of the odds to respond among patients receiving ENLIGHT-matched treatments vs. the odds to respond among patients whose treatments were not ENLIGHT-matched. Patients were considered ENLIGHT-matched if their EMS scores were greater or equal to a threshold value of 0.54. This threshold was determined already in the original ENLIGHT publication on independent data and was kept fixed here. Using this predefined threshold, we observe that the OR of ENLIGHT-DeepPT is higher than 1 for all datasets, though this was not statistically significant for the PARPi and ALKi datasets, probably due to their small sample sizes (FIG. 14A). This demonstrates that patients receiving ENLIGHT-matched treatments indeed had a higher chance to respond.

Complementing the OR measure, which is of major translational interest as it quantifies the performance at a specific decision threshold, FIG. 14B further depicts the prediction performance of ENLIGHT-DeepPT via a complementary measure, the average precision (AP). AP is a measure of the precision of a prediction model across the entire range of thresholds. For a classifier to be of merit, its AP should be higher than the overall response rate (ORR), which denotes the fraction of responders observed in each cohort. Reassuringly, the AP of ENLIGHT-DeepPT well exceeds the ORR for all five datasets, testifying to its broader predictive power beyond that quantified by the OR.

Turning to an aggregate analysis of the performance of ENLIGHT-DeepPT, by analysing all patients together in a simulated “basket” trial in which each patient receives a different treatment (n=234), the OR of ENLIGHT-DeepPT is 2.44 ([1.36,4.38] 95% CI, left bar in FIG. 14A), significantly higher than 1 (p=0.002, Fisher's exact test), and its precision is 47.8%, a 43.5% increase compared to the overall response rate of 33.3% (p=1.28×10\hat{ }(−6), one sided proportion test). The AP is 0.48 (left bar, FIG. 14B), significantly higher than the baseline response rate (p=0.0003, one sided permutation test).

### A Comparison of ENLIGHT-DeepPT to a Direct Supervised Approach

One of the advantages of ENLIGHT's unsupervised approach is that it does not rely on data labelled with response to treatment data which is usually scarce. Such data is required for training supervised models, which in theory, given sufficiently large datasets, are expected to yield higher performance on unseen datasets than unsupervised methods like ENLIGHT. The question remains whether such supervised methods are advantageous for realistically small datasets as studied in this work. To compare the performance of our two-step indirect model with a direct supervised model, we trained the same computational deep learning pipeline as the one used in DeepPT on H&E slides and their corresponding response data on each of the five evaluation datasets described above, except that we replaced the regression component with a classification component. We used the same training strategy that has been widely applied previously in the literature for direct H&E slide-based, and termed this the Direct Supervised model. Due to the lack of independent treatment data for training, we applied leave-one-out cross-validation (LOOCV) to evaluate the performance of the direct supervised models. This in-cohort training gives an inherent advantage to the Direct Supervised approach over ENLIGHT-DeepPT, which was not exposed to these datasets at all. A comparison between the Direct Supervised model and ENLIGHT-DeepPT is given in FIG. 14C-14E. Since no tuning data was available to calibrate a single threshold for the Direct Supervised methods as was done in Dinstag et al. Med. 4(1):15-30.e8), we calculated the OR of the Direct Supervised for all patients (n=234) on all possible thresholds. FIG. 14C presents the OR as a function of the coverage (the fraction of patients with scores above a specific threshold). We compared these values to the OR of ENLIGHT-DeepPT at the clinical decision threshold established in Dinstag et al. Med. 4(1):15-30.e8. based on measured RNA levels (0.54, red dashed line). Surprisingly, no threshold on the Direct Supervised values yields an OR that surpasses the OR of ENLIGHT-DeepPT at its predetermined clinical decision threshold. In addition, we calculated ENLIGHT-DeepPT's OR at various possible EMS thresholds and compared the results at thresholds that yield the same coverage in both ENLIGHT-DeepPT and Direct Supervised (FIG. 14D). Finally, FIG. 14E compares the average precision (which is threshold independent) of the two models. Remarkably, the overall performance of ENLIGHT-DeepPT, being an unsupervised method, as measured by OR and AP, is comparable with that of the supervised classifiers trained and predictive only for specific treatments, and in some cases ENLIGHT-DeepPT even outperforms them. Moreover, training in-cohort, as was done here for the supervised methods due to lack of sufficient data, has a clear risk of overfitting.

Ideally, an external dataset is required to study the generalizability of the model. Among the datasets of this study, this was possible only for Trastuzumab which appeared in more than one dataset. When we tested the model trained on one Trastuzumab dataset on the other set and vice versa we saw low generalizability: the AP went down from 0.52 in LOOCV for Trastuzumab1 to 0.27 when this model was tested on Trastuzumab2, and from 0.5 to 0.43 in the other direction. This suggests that the results obtained for the supervised models may be overfitted. Clearly, for cases where large data exists, a supervised method can outperform ENLIGHT-DeepPT. In contrast, ENLIGHT-DeepPT is unsupervised, and the results presented here testify to its potential generalizability. In addition, any supervised model can only be obtained for drugs with coupled H&E and response data (4 drugs in this study), while ENLIGHT-DeepPT can produce predictions to virtually any targeted treatment.

For one of the datasets (Trastuzumab1), RNA sequencing of the tumor gene expression was also available and was previously analyzed by ENLIGHT in Dinstag et al. Med. 4(1):15-30.e8. FIGS. 14f and 14g compare the predictive performance using ENLIGHT-DeepPT scores to that using ENLIGHT scores calculated based on the measured expression values (denoted ENLIGHT-actual). Using the previously established threshold of 0.54, the OR of ENLIGHT-DeepPT is 3.2, which is lower than the OR of 6.95 obtained by ENLIGHT-actual, but still significantly higher than expected by chance (p=0.02, test for OR >1). The positive predictive value (PPV) (also known as precision) of ENLIGHT-DeepPT was 53.3%, slightly higher than but not significantly different from the PPV of 52% when using ENLIGHT-actual, and 80% higher than the basic ORR of 29.7% observed in this study. However, the sensitivity (the fraction of responders correctly identified) of ENLIGHT-DeepPT is markedly lower than that of ENLIGHT-actual, 42.1% vs. 68. %.

### A Comparison of ENLIGHT-DeepPT to Other Predictive Models

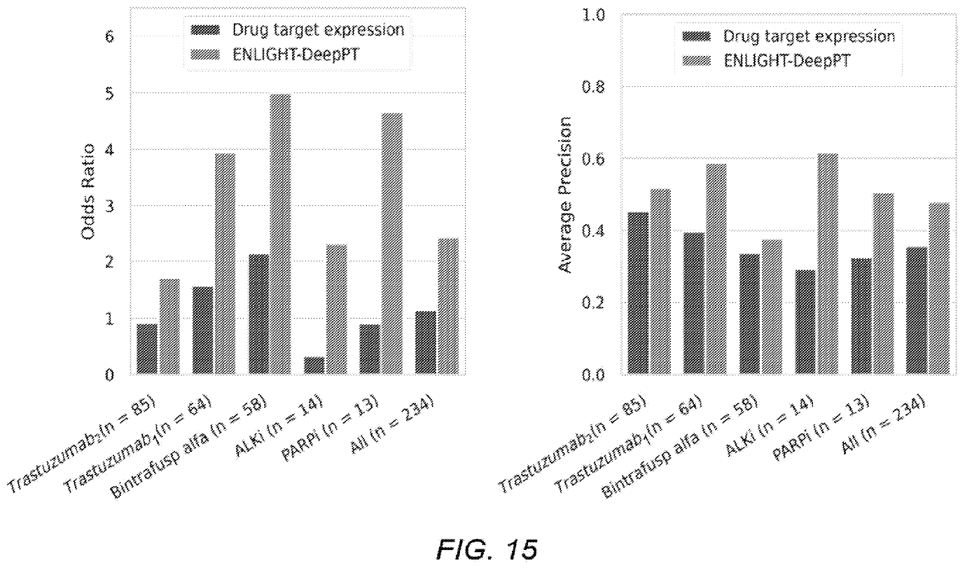

Finally, we sought to compare ENLIGHT-DeepPT to other predictive models for drug response. For the drugs analyzed in this study, the only available mRNA-based model for response is the multi-omic machine learning predictor that uses DNA, RNA and clinical data, published by Sammut et al. denoted here as Sammut-ML. This model was based on in-cohort supervised learning to predict response to chemotherapy with or without trastuzumab among HER2+ breast cancer patients. FIG. 14G compares ENLIGHT-actual and ENLIGHT-DeepPT performance to Sammut ML. In both analyses, we applied all methods to the same patient group of 56 patients for whom all relevant data was available (RNAseq, H&E slide, DNAseq and clinical features). To systematically compare between the predictors across a wide range of decision thresholds, and since Sammut et al. did not derive a binary classification threshold, we used AP as the comparative rod here. As can be seen, all methods have quite comparable predictive power, with ENLIGHT-DeepPT having the highest AP (difference not statistically significant). Importantly, using only H&E slides without need for RNA or DNA data or other clinical features has an invaluable practical advantage. Notably, the predictions for Trastuzumab1 were made on fresh frozen tissue slides, which differ considerably from FFPE samples used to train the DeepPT model, testifying to the robustness of DeepPT and ENLIGHT. To complement this analysis, we show that ENLIGHT-DeepPT outperforms a model that uses only the predicted expression of the drug targets as predictors of response (FIG. 15).

### DISCUSSION

Our study demonstrates that combining DeepPT, a novel deep learning framework for predicting gene expression from H&E slides, with ENLIGHT, a published unsupervised computational approach for predicting patient response from pre-treated tumor transcriptomics, could be used to form a new ENLIGHT-DeepPT approach for H&E-based prediction of clinical response to a host of targeted and immune therapies. We began by showing that DeepPT significantly outperforms the current state-of-the-art method in predicting mRNA expression profiles from H&E slides. Then, we showed that the aggregate signal from multiple genes can overcome weak correlation at the individual gene level. Finally, and most importantly, ENLIGHT-DeepPT successfully predicts the true responders in several clinical datasets from different indications, treated with a variety of targeted drugs directly from the H&E images, demonstrating its potential clinical utility throughout. Notably, its prediction accuracy on these datasets is on par with that of direct predictors from the images, as is the current practice, even though the latter have been trained and tested in a cross validation in-cohort manner.

Combining DeepPT with ENLIGHT is a promising approach for predicting response directly from H&E slides because it does not require response data on which to train. This is a crucial advantage compared to the more common practice of using response data to train classifiers in a supervised manner. Indeed, while sources like TCGA lack response data that would enable building a supervised predictor of response to targeted and immune treatments, applying ENLIGHT to predicted expression has successfully enabled the prediction of response to four different treatments in five datasets spanning six cancer types with considerable accuracy, without the need for any treatment data for training. While supervised models can only be obtained for drugs with available H&E and response data, ENLIGHT-DeepPT can produce predictions to virtually any targeted treatment, and importantly, including ones in early stages of development where such training data is still absent.

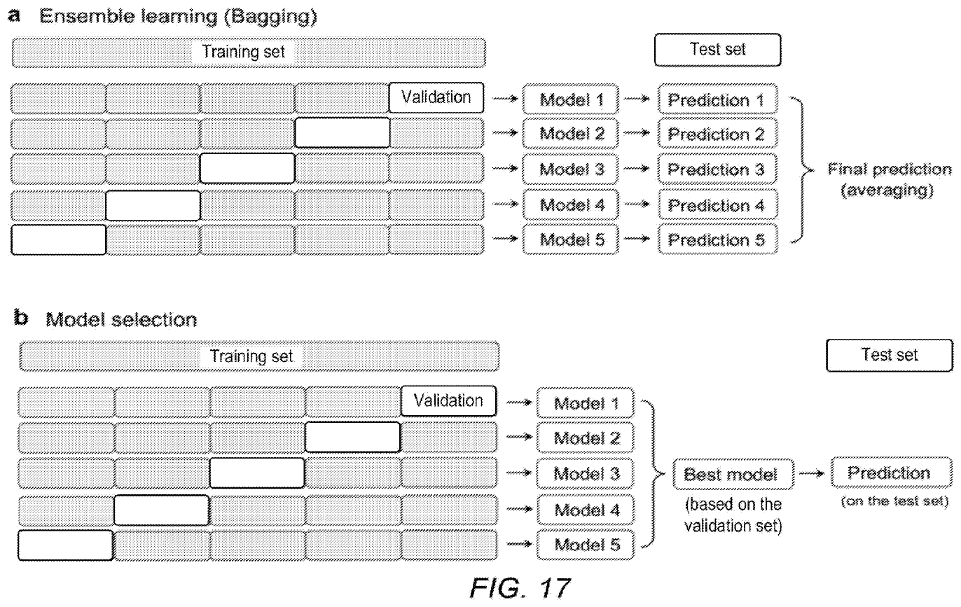

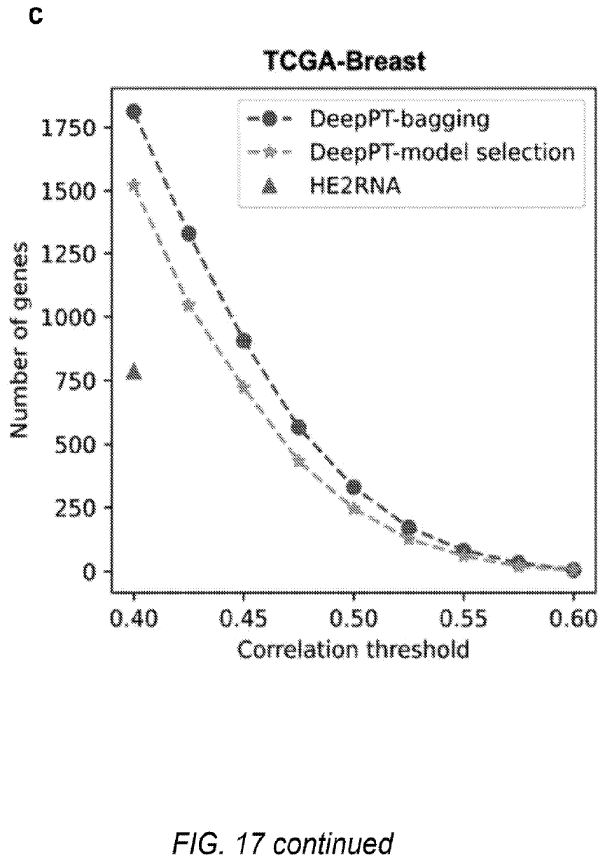

DeepPT is fundamentally different from previous computational pipelines for gene expression prediction both in its model architecture and in its training strategy. We attribute its superior performance to four key innovations: (i) All previous studies fed output from the conventional pre-trained CNN model (trained with natural images from ImageNet database) directly into their regression module, whereas we added an auto-encoder to re-train the output of the pre-trained CNN model. This helps to exclude noise, avoid overfitting and reduce the computational demands. (ii) Our regression module is an MLP model in which the weights from the input layer to the hidden layer are shared among genes. This architecture enables the model to exploit the correlations between the expression of the genes. (iii) We trained together sets of genes with similar median gene expression values; doing so further implements a form of multitask learning and prevents the model from focusing on only the most highly expressed genes. (iv) We performed ensemble learning by taking the mean predictions across all models. This further improves the prediction accuracy quite significantly (FIG. 17).

DeepPT can be broadly applied to all cancer types for which H&E slides and coupled mRNA data are available for training; however, similar to many other deep learning models, it requires a considerable number of training samples comprising matched imaging and gene expression. An interesting direction for future work would be to apply transfer learning between cohorts, to improve the predictive performance in cancer types with yet small training cohorts. In other words, it might be possible to train the model on large TCGA cohorts such as breast and lung cancer, then fine tune it for generating predictions for cancer types with smaller TCGA cohorts such as melanoma or ovarian cancer.

A notable finding of this study is the robustness of response predictions based on H&E slides when combining DeepPT and ENLIGHT. First, despite the inevitable noise introduced by the prediction of gene expression, the original ENLIGHT GI networks, designed to predict response from measured RNA expression, worked well as-is in predicting response based on the DeepPT-predicted expression. In fact, when restricting the GI networks to include only significantly predicted genes, the results are not improved. Second, though DeepPT was trained using FFPE slides, it generalized well and could be used as-is to predict expression values from FF slides. This demonstrates the applicability of DeepPT for predicting RNA expression either from FF or from FFPE slides. Nevertheless, as promising as the results presented here are, they should of course be further tested and expanded upon by applying the generic pipeline presented here to many more cancer types and treatments.

Developing a response prediction pipeline from H&E slides, if reasonably accurate and further carefully tested and validated in clinical settings, could obviously be of utmost benefit, as NGS results often take 4-6 weeks after initiation to return a result. Many patients who have advanced cancers require treatment immediately, and this method can potentially offer treatment options within a shorter time frame. Moreover, obtaining H&E images can be done at relatively low cost, compared to the expenses incurred by NGS. Increasing efforts to harness the rapid advances in deep learning are likely to improve precision oncology approaches, including by leveraging histopathology images. Given its general and unsupervised nature, we are hopeful that ENLIGHT-DeepPT may have considerable impact, making precision oncology more accessible to patients in Low-and middle-income countries (LMICs), in under-served regions and in other situations where sequencing is less feasible. Specifically, affordable cancer diagnostics is critical in LMICs since their limited access to cancer diagnostics is a bottleneck for effectively leveraging the increasing access to cancer medicine. While promising, one should of course cautiously note that the results presented in this study await a broader testing and validation in carefully designed prospective studies before they may be applied in the clinic. We are hopeful that the results presented here will expedite such efforts by others going forward.

TABLE 1
Number of slides and number of patients for each cohort.

| Cohort | #slides | #patients |
|---|---|---|
| TCGA-Breast | 1,106 | 1,043 |
| TCGA-Lung | 1,018 | 927 |
| TCGA-Brain | 1,015 | 574 |
| TCGA-Kidney | 859 | 836 |
| TCGA-Colorectal | 514 | 510 |
| TCGA-Prostate | 438 | 392 |
| TCGA-Gastric | 433 | 410 |
| TCGA-Head&Neck | 430 | 409 |
| TCGA-Cervical | 261 | 252 |
| TCGA-Pancreas | 195 | 175 |
| TransNEO-Breast | 160 | 160 |
| NCI-Brain | 226 | 226 |

TABLE 2
Details of the 5 datasets analyzed using ENLIGHT-DeepPT.

| | | | Background | | # | Responders |
|---|---|---|---|---|---|---|
| Name | Indication | Drug | Therapy | N | responders | criteria |
| PARPi | Pancreas | Olaparib or | | 13 | 5 | OS >36 |
| | | | | | | months |
| Trastuzumab₁ | Breast | Trastuzumab | Chemotherapy | 64 | 18 | pCR |
| Trastuzumab₂ | Breast | Trastuzumab | Chemotherapy | 85 | 36 | pCR |
| Bintrafuspalfa | Lung, | Bintrafusbalfa | | 58 | 13 | CR, PR |
| | Head& | | | | | |
| | Neck, | | | | | |
| | Cervical | | | | | |
| ALKi | Lung | Alectinib or | | 14 | | >18 months |
| | | Crizotinib | | | | PFS |

OS—overall survival;
pCR—pathological complete response;
RD—residual disease;
CR—complete response;
PR—partial response;
RCB—residual cancer burden.

### Example 2—Clinical Trial Design with ENLIGHT-DeepPT Algorithm

The present example is aimed at testing ENLIGHT-DeepPT as a tool for clinical trial design (CTD), where the goal is to a-priori exclude non-responding patients in the best possible manner. The fact that ENLIGHT is essentially an unsupervised prediction method, that is not trained on treatment outcome data, enables biomarker discovery before any human data accumulates. This is especially important for drugs in the approval process, before clinical trial data or real-world data are generated. The algorithm ENLIGHT was previously shown to enable near optimal exclusion of non-responding patients in CTD based on measured mRNA. The present study investigates whether CTD can be based on H&E slides. The tested case concerns Bintrafusp Alfa, a drug that has yet to be approved.

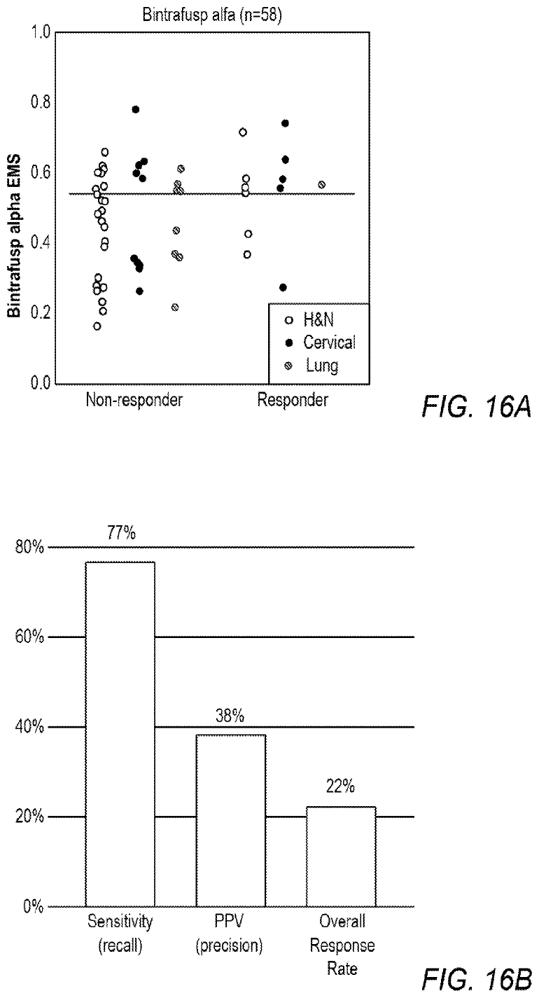

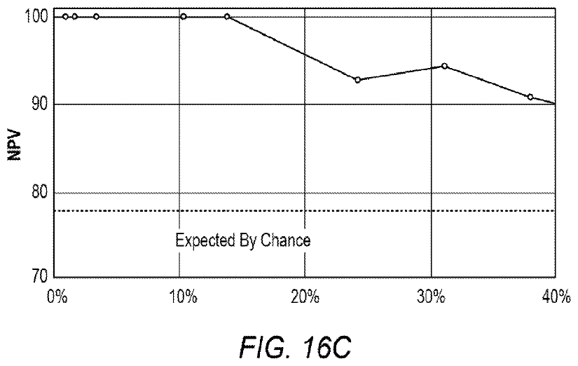

The ENLIGHT Matching Score (EMS), which evaluates the match between patient and treatment was found predictive of response to Bintrafusp Alfa (FIGS. 16A and 16B). It is important to emphasize that in the case, the EMS was calculated based on mRNA data that was predicted using the said method. Next, the proportion of true non-responders among those predicted not to respond (NPV) as a function of the percent of patients excluded, where patients are excluded by order of increasing EMS, was calculated (FIG. 16C). As desired, ENLIGHT-DeepPT's NPV curve is considerably higher than the NPV expected by chance, i.e., the percentage of non-responders. The response rate in the remaining cohort after excluding the bottom relevant percentile of patients based on low EMS was calculated. The dotted-dashed line represents the limit performance of an optimal “all-knowing” classifier that excludes only non-responders, retaining only true responders (FIG. 16D). As evident, excluding patients based on the EMS calculated directly from H&E slides, increases the response rate among the remaining patients (middle, solid line), and reach near optimal exclusion (see the dotted-dashed line).

### Example 3—DeepPT Reconstructs Prognostic Signatures in TCGA

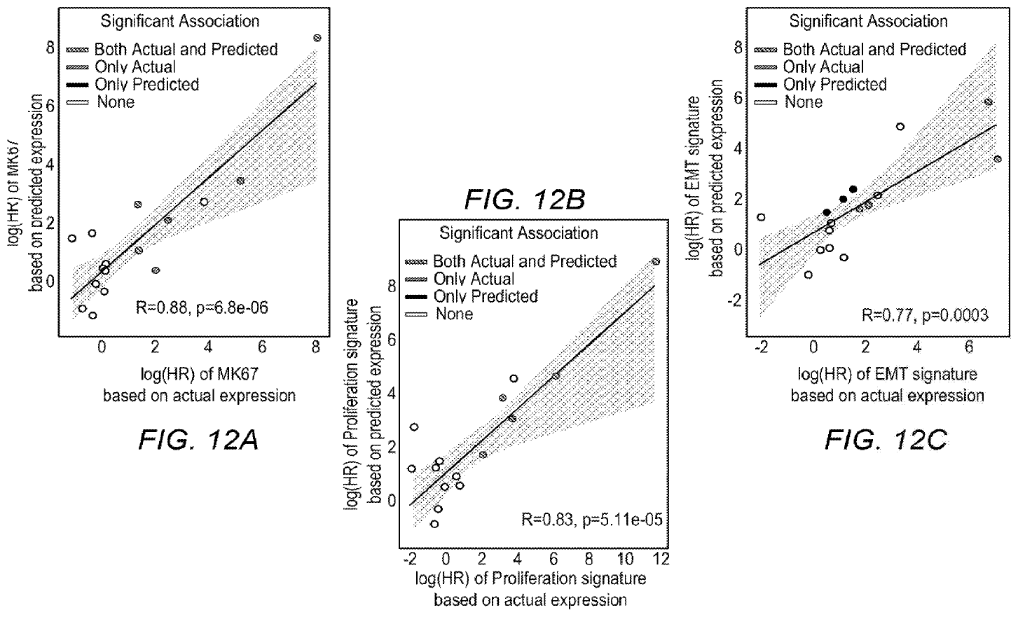

The observation that genes that promote proliferation and metastasis, which are well known prognostic markers, are specifically well predicted by DeepPT, led us to explore how well these prognostic markers predicted patient survival, when calculated over the gene expressions predicted by DeepPT. To this end, we calculated the survival association of three mRNA proliferation signatures known to be linked to cancer progression and poor prognosis using mRNA predicted from H&E slides by DeepPT based on TCGA patients data. These signatures include (a) the expression of the MK67 gene, a well-known marker for cell proliferation, (b) the proliferation index derived in Whitfield et al. and (c) an epithelial to mesenchymal transition (EMT) signature from MsigDB, associated with the formation and progression of metastasis. For each patient in each TCGA cohort, we calculated a signature score that is the mean gene-wise ranked gene expression across the genes of the signature. We then tested the correlation between signature scores derived from the predicted and the actual gene expression, and the association of each of these signature scores to patient survival using a cox proportional hazard at the cohort level (FIGS. 12A-12C). First, all three signature scores exhibit a significant correlation between their actual and DeepPT predicted expressions (top row). Importantly, the multi-gene EMT and proliferation signatures exhibit higher correlations (0.396 and 0.421) than the expression of the single MK67 gene (0.327), and a higher correlation than the individual genes that constitute these signatures (mean gene wise correlation of 0.364 and 0.281 across each signature, respectively). Notably, even though the correlation of the scores themselves are in the medium range, DeepPT reconstructs the prognostic value of the signatures fairly faithfully: the correlation between the hazard ratio of each signature between those computed based on the actual and predicted expressions across the TCGA cancer types is very high (0.77-0.88, FIG. 12A-12C). These results testify that the ensemble of multiple genes yields a higher correlation when combined, as expected, and remarkably, the prognostic value of these signatures is well retained by DeepPT.

It will be recognized that examples, embodiments, modifications, options, etc., described herein are to be construed as inclusive and non-limiting, i.e., two or more examples, embodiments, modifications, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1. A method of predicting a gene expression profile in a subject based on a histopathological image using an artificial neural network (ANN), the method comprising:
(a) providing an artificial neural network (ANN) comprising a feature extraction module, an auto-encoder, and a multi-layer perceptron (MLP),
wherein said extraction module is configured to receive an input signal representing a histopathological image, to extract a predetermined number of features from said input signal, and to transmit said extracted features to said autoencoder;
wherein said autoencoder is configured to receive the features extracted from the extraction module, to compress the number of said extracted features, and to present said compressed features to said MLP;
wherein said MLP is configured to receive the compressed features from the autoencoder in an input layer of neurons and to provide an output in an output layer of neurons, said output representing a gene expression profile;
wherein said ANN is trained to predict a gene expression profile from a histopathological image;
(b) providing said histopathological image to said ANN;
(c) obtaining a predicted gene expression profile of the subject with said ANN.

2. A method of predicting the response to a therapeutic intervention in a subject having a pathological condition, the method comprising the steps of:
(a) providing one or more histopathological images from said subject;
(b) obtaining a predicted gene expression profile of said subject according to the method of claim 1;
(c) determining a treatment score from the predicted gene expression profile for the subject with a SELECT or an ENLIGHT algorithm, wherein the treatment score is indicative of a likelihood of successfully treating said subject with said therapeutic intervention; and
(d) predicting the response to said therapeutic intervention in said subject based on said treatment score.

3. A method of selecting a therapeutic intervention for a subject in need thereof, the method comprising the steps of:
(a) providing a plurality of therapeutic interventions;
(b) predicting the response to each of said therapeutic interventions in said subject according to method of claim 2; and
(c) selecting the therapeutic intervention with the treatment score that is indicative of the highest likelihood of successfully treating the subject.

4. A method of treating a subject in need thereof, the method comprising:
(a) selecting a therapeutic intervention for said subject according to the method of claim 3; and
(b) treating the subject with the therapeutic intervention selected in step (a).

5. A method of ranking a set of subjects having a pathological condition based on the likelihood of successfully treating each subject with a therapeutic intervention, the method comprising the steps of:
(a) providing a set of subjects having a pathological condition;
(b) providing one or more histopathological images of each of said subjects;
(c) predicting the response to said therapeutic intervention in each subject according to the method of claim 2; and
(d) ranking each subject in the set of subjects based on the probability of successfully treating that subject with said therapeutic intervention.

6. The method of claim 5, wherein the set of subjects are prospective participants in a clinical trial.

7. A method for designing a clinical trial for a therapeutic intervention, the method comprising the steps of:
(a) providing a set of subjects having a pathological condition;
(b) predicting the response of each of said subjects to said therapeutic intervention according to the method of claim 2;
(c) selecting for said clinical trials the subjects having a treatment score higher than a predetermined value.

8. A method of monitoring a subject having a pathological condition who is or will be undergoing a therapeutic intervention, the method comprising the steps of:
(a) predicting the response to said therapeutic intervention in said subject according to the method of claim 2;
(b) repeating steps (a) a plurality of times to monitor the course of treatment.

9. The method of any one of claims 1-8, wherein said ANN is trained to predict a gene expression profile from a histopathological image by a training method comprising:
(a) providing a training set of histopathological images taken from patients having a pathological condition;
(b) providing a training set of gene expression profiles, wherein each gene expression profile from said training set of gene expression profiles corresponds to one of the histopathological images from said training set of histopathological images;
(c) presenting to said feature extraction module a first input signal representing a histopathological image from the training set of histopathological images and presenting to said output layer of said MLP a second input signal representing the corresponding gene expression profile;
(d) adjusting the weights of said ANN; and
(e) repeating steps (c) and (d) for all histopathological images and their corresponding gene expression profile of said training sets;
(f) repeating steps (c)-(e) a number of epochs.

10. The method of claim 9, wherein each gene expression profile from said training set is divided into subsets of genes characterized by similar median expression, and wherein said ANN is trained with each subset separately.

11. The method according to claim 9 or 10, wherein the training comprises a hold-out method.

12. The method according to any one of claims 9-11, wherein the training comprises k-fold cross-validation.

13. The method of any one of claims 1-12, wherein said histopathological image is divided into sub-images, and wherein each sub-image is presented separately to the input layer of said feature extraction module.

14. The method of claim 13, wherein the predicted gene expression of a histopathological image is calculated by pooling the gene expression profile predicted for each of said sub-images.

15. The method of claim 14, wherein said pooling comprises the average or the median of said output produced by each of said sub-images.

16. The method of any one of claims 1-15, wherein said ANN comprises a convolutional neural network (CNN).

17. The method according to claim 16, wherein the CNN comprises a ResNet50 CNN.

18. The method according to any one of claims 1-17, wherein the number of neurons in the input layer of the MLP is equal to the number of features compressed by the autoencoder.

19. The method according to any one of claims 1-18, wherein the MLP comprises a hidden layer, and the number of neurons in said hidden layer is less than or equal to the number of features compressed by the autoencoder.

20. The method according to any one of claims 1-19, wherein the number of neurons in the output layer of the MLP is equal to the number of genes of said gene expression profile.

21. The method according to any one of claims 1-20, wherein the number of features compressed by the autoencoder is no greater than about half, or no greater than about one quarter of the number of features extracted by the ANN.

22. The method according to any one of claims 1-21, wherein the ANN is configured to extract 2,048 features, and the autoencoder is configured to extract 512 features.

23. The method according to any one of claims 1-22, wherein said histopathological images are whole slide images of stained tissue.

24. The method according to claim 23, wherein said tissue is stained with haematoxylin and eosin.

25. The method according to any one of claims 1-24, wherein said gene expression profile comprises genes expressed above a predetermined threshold.

26. The method of claim 21, wherein said genes above a predetermined threshold are identified using edgeR.

27. The method according to any one of claims 1-26, wherein the pathological condition is a cancer.

28. The method according to claim 27, wherein the cancer is selected from a group including breast, lung, brain, kidney, colorectal, prostate, gastric, head and neck, cervical, and pancreas cancer.

29. A method for identifying a pair of genes comprising a synthetic lethality (SL), synthetic rescue (SR), or synthetic dosage lethality (SDL) interaction, using a depletion model, said method implemented by a computer processor executing program instructions comprising:
(a) obtaining gene expression profiles from a set of subjects according to the method of claim 1;
(b) transforming expression data relating to each of two genes from said gene expression profiles, through ranking, thereby producing two uniform transformed distributions in the range [0, 1];
(c) calculating a resulting joint expression distribution for the gene pair, having uniform marginal distributions;
(d) identifying a parametric family of distributions comprising a shape parameter wherein said shape parameter determines the degree of corner depletion or enrichment for one or more of the corners in the joint distribution, and fitting said shape parameter to said joint expression data; and
(e) calculating a value of a best-fitting shape parameter as an indication of the genetic interaction between said two genes.

30. A method for identifying a pair of genes comprising a synthetic lethality (SL), synthetic rescue (SR), or synthetic dosage lethality (SDL) interaction, using a parametric survival model, said method implemented by a computer processor executing program instructions comprising:
(g) obtaining gene expression profiles from a set of subjects according to the method of claim 1;
(h) Transforming expression data relating to each of two genes from a population, thereby producing two uniform transformed distributions in the range [0, 1];
(i) Calculating a resulting joint expression distribution for the gene pair, having uniform marginal distributions;
(j) Identifying a theoretical distribution function D that approximates the joint distribution of the transformed expression levels of said pair of genes;
(k) Calculating a covariate value (c(p)) for a given patient in a population of patients (cohort P), by calculating a ratio of the density of said theoretical distribution function D at joint expression values (x,y) of said gene pair, to a maximal D density value or a minimal D density value, across a full joint distribution space; and
(l) Assessing a correlation between (i) a set of covariates C:={c{p)| p in P} obtained in “c” for said patients of cohort P; and (ii) survival of the patients in said cohort, as an assessment of the strength of the corresponding genetic interaction between said gene pair.

31. The method of claim 29 or 30, wherein said synthetic rescue (SR) comprises synthetic rescue DD (SR-DD) or synthetic rescue DU (SR-DU).

32. A method for identifying a drug target candidate, the method comprising identifying genes comprising SL, SR, or SDL interactions according to the method of any one of claims 29-31; wherein a gene, or a protein encoded thereof, comprising a number of SL, SR, or SDL interactions above a predetermined threshold, is as a drug target candidate.

33. A method of predicting the efficiency of novel therapeutic combinations, the method comprising identifying pair of genes comprising SL, SR, or SDL interactions according to the method of any one of claims 29-31; wherein a therapeutic combinations targeting a pair of genes comprising SL, SR, or SDL interactions is predicted to be efficient.

34. A method of training an artificial neural network (ANN) to predict a gene expression profile of a patient having a pathological condition, the method comprising:
(a) providing an artificial neural network (ANN) comprising a feature extraction module, an auto-encoder, and a multi-layer perceptron (MLP),
wherein said extraction module is configured to receive an input signal representing a histopathological image, to extract a predetermined number of features from said input signal, and to transmit said extracted features to said autoencoder;
wherein said autoencoder is configured to receive the features extracted from the extraction module, to compress the number of said extracted features, and to present said compressed features to said MLP;
wherein said MLP is configured to receive the compressed features from the autoencoder in an input layer of neurons and to provide an output in an output layer of neurons, said output representing a gene expression profile;
(b) providing a training set of histopathological images taken from patients having said pathological condition;
(c) providing a training set of gene expression profiles, wherein each gene expression profile from said training set of gene expression profiles corresponds to one of the histopathological images from said training set of histopathological images;
(d) presenting to said feature extraction module a first input signal representing a histopathological image from the training set of histopathological images and presenting to said output layer of said MLP a second input signal representing the corresponding gene expression profile;
(e) adjusting the weights of said ANN; and
(f) repeating steps (d) and (e) for all histopathological images and their corresponding gene expression profile of said training sets;
(g) repeating steps (d)-(f) a number of epochs.

35. The method of claim 34, wherein each gene expression profile from said training set is divided into subsets of genes characterized by similar median expression, and wherein said ANN is trained with each subset separately.

36. The method according to claim 34 or 35, wherein the training comprises a hold-out method.

37. The method according to any one of claims 34-36, wherein the training comprises k-fold cross-validation.

38. The method of any one of claims 34-37, wherein said histopathological image is divided into sub-images, and wherein each sub-image is presented separately to the input layer of said feature extraction module.

39. The method of claim 38, wherein said sub-images are non-overlapping.

40. The method according to any one of claim 38 or 39, wherein sub-images containing more than half of the pixels with a weighted gradient magnitude smaller than a certain threshold are excluded.

41. The method according to any one of claims 38, wherein the predicted gene expression of a histopathological image is calculated by pooling the gene expression profile predicted for each of said sub-images.

42. The method of claim 41, wherein said pooling comprises the average or the median of said output produced by each of said sub-images.

43. The method of any one of claims 34-42, wherein said ANN comprises a convolutional neural network (CNN).

44. The method according to claim 43, wherein the CNN comprises a ResNet50 CNN.

45. The method according to any one of claims 34-44, wherein said histopathological images are whole slide images of stained tissue.

46. The method according to claim 45, wherein the tissue is stained with haematoxylin and eosin.

47. The method according to any one of claims 34-46, wherein said gene expression profile comprises genes expressed above a predetermined threshold.

48. The method of claim 47, wherein said genes expressed above a predetermined threshold are identified using edgeR.

49. The method according to any one of claims 34-48, wherein the pathological condition is a cancer.

50. A system for predicting a gene expression profile based on a histopathological image, the system comprising:
an artificial neural network (ANN), comprising a feature extraction module, an auto-encoder, and a multi-layer perceptron (MLP),
wherein said extraction module is configured to receive an input signal representing a histopathological image, to extract a predetermined number of features from said input signal, and to transmit said extracted features to said autoencoder;
wherein said autoencoder is configured to receive the features extracted from the extraction module, to compress the number of said extracted features, and to present said compressed features to said MLP;
wherein said MLP is configured to receive the compressed features from the autoencoder in an input layer of neurons and to provide an output in an output layer of neurons, said output representing a gene expression profile;
wherein said ANN is trained to predict a gene expression profile from a histopathological image.

51. A system for predicting the response to a therapeutic intervention in a subject having a pathological condition, the system comprising the system for predicting a gene expression profile of claim 50;
(a) wherein the system for predicting the response to a therapeutic intervention is configured to determine a treatment score from the predicted gene expression profile for the subject with a SELECT or an ENLIGHT algorithm, wherein the treatment score is indicative of a likelihood of successfully treating the subject with the therapeutic intervention; and
(b) wherein the system for predicting the response to a therapeutic intervention is configured to predict the response to said therapeutic intervention in said subject based on said treatment score.

52. A system for selecting a therapeutic intervention for a subject in need thereof, the system comprising the system for predicting the response to a therapeutic intervention of claim 51;
(a) wherein the system for selecting a therapeutic intervention is configured to provide a prognosis score for a plurality of therapeutic interventions; and
(b) wherein the system for selecting a therapeutic intervention is configured to select the therapeutic intervention with the treatment score that is indicative of the highest likelihood of successfully treating the subject.

53. A system for ranking a set of subjects having a pathological condition based on the likelihood successfully treating each subject with a therapeutic intervention, the system comprising the system for predicting the response to a therapeutic intervention of claim 51;
(a) wherein the system for ranking a set of subjects is configured to predict the response to a therapeutic intervention for each subject treated with said therapeutic intervention;
(b) wherein the system for ranking a set of subjects is configured to rank each subject from the set of subjects based on the probability of successfully treating that subject with said therapeutic intervention.

54. A system for designing a clinical trial for a therapeutic intervention, the system comprising the system for predicting the response to a therapeutic intervention of claim 51;
(a) wherein the system for designing a clinical trial is configured to receive information regarding a set of subjects having a pathological condition;
(b) wherein the system for designing a clinical trial is configured to select for said clinical trial subjects having a treatment score higher than a predetermined value.

55. A system for monitoring a subject having a pathological condition who is or will be undergoing a therapeutic intervention, the system comprising the system for predicting the response to a therapeutic intervention of claim 51; wherein the system for monitoring a subject is configured to predict the response to said therapeutic intervention in said subject a plurality of times.

56. The system of any of claims 50-55, wherein said ANN is trained to predict a gene expression profile from a histopathological image by a training method comprising:
(a) providing a training set of histopathological images taken from patients having said pathological condition;
(b) providing a training set of gene expression profiles, wherein each gene expression profile from said training set of gene expression profiles corresponds to one of the histopathological images from said training set of histopathological images;
(c) presenting to said feature extraction module a first input signal representing a histopathological image from the training set of histopathological images and presenting to said output layer of said MLP a second input signal representing the corresponding gene expression profile;
(d) adjusting the weights of said ANN; and
(e) repeating steps (c) and (d) for all histopathological images and their corresponding gene expression profile of said training sets;
(f) repeating steps (c)-(e) a number of epochs.

57. The system of any one of claims 50-56, further comprising a module configured to receive said histopathological image, to divide it into sub-images, and to present each sub-image to the input layer of said feature extraction module.

58. The system of claim 57, further comprising a module configured to receive the output produced by each of said sub-images, pooling said outputs, and calculating the predicted gene expression of each gene based on said pooling.

59. The system of claim 58, wherein said pooling comprises averaging or calculating the median value of said output produced by each of said sub-images.

60. The system according to any one of claims 50-59, wherein said ANN comprises a convolutional neural network (CNN).

61. The system of claim 60, wherein the CNN comprises a ResNet50 CNN.

62. The system according to any one of claims 50-61, wherein the number of neurons in the input layer of the MLP is equal to the number of features compressed by the autoencoder.

63. The system according to any one of claims 50-62, wherein the MLP comprises a hidden layer, and the number of neurons in said hidden layer is less than or equal to the number of features compressed by the autoencoder.

64. The system according to any one of claims 50-63, wherein the number of neurons in the output layer of the MLP is equal to the number of genes of said gene expression profile.

65. The system according to any one of claims 50-64, wherein said histopathological images are whole slide images of stained tissue.

66. The system according to claim 65, wherein the tissue is stained with haematoxylin and eosin.

67. The system according to any one of claims 50-66, wherein said gene expression profile comprises genes expressed above a predetermined threshold.

68. The system of claim 67, wherein said genes expressed above a predetermined threshold are identified using edgeR.

69. The system according to any one of claims 50-68, wherein the pathological condition is a cancer.

70. A system for training an artificial neural network (ANN) to predict gene expression profile of a patient having a pathological condition, the system comprising:
an artificial neural network (ANN), comprising a feature extraction module, an auto-encoder, and a multi-layer perceptron (MLP),
wherein said extraction module is configured to receive an input signal representing a histopathological image, to extract a predetermined number of features from said input signal, and to transmit said extracted features to said autoencoder;
wherein said autoencoder is configured to receive the features extracted from the extraction module, to compress the number of said extracted features, and to present said compressed features to said MLP;
wherein said MLP is configured to receive the compressed features from the autoencoder in an input layer of neurons and to provide an output in an output layer of neurons, said output representing a gene expression profile;
wherein said system is configured to
(a) receive a training set of histopathological images taken from patients having said pathological condition;
(b) receive a training set of gene expression profiles, wherein each gene expression profile from said training set of gene expression profiles corresponds to one of the histopathological images from said training set of histopathological images;
(c) present to said feature extraction module a first input signal representing a histopathological image from the training set of histopathological images and presenting to said output layer of said MLP a second input signal representing the corresponding gene expression profile;
(d) adjust the weights of said ANN; and
(e) repeat steps (c) and (d) for all histopathological images and their corresponding gene expression profile of said training sets;
(f) repeat steps (c)-(e) a number of epochs.

71. The system according to claim 70, wherein said ANN comprises a convolutional neural network (CNN).

72. The system according to claim 71, wherein the CNN comprises a ResNet50 CNN.

73. The system according to any one of claim 70 or 71, wherein each of said histopathological images is divided into sub-images, and each sub-image is used as the input to the feature extraction module to train the ANN.
